(19) United States
(12) Patent Application Publication
(10) Pub. No.: US 2025/0176479 A1
(43) Pub. Date: Jun. 5, 2025

(54) METHODS AND SYSTEMS FOR MODIFYING AERIAL MYCELIUM GROWTH AND GROWTH TRAJECTORIES IN SITU, AND AERIAL MYCELIUM-BASED MATERIALS PRODUCED THEREBY THROUGH IN SITU GROWTH INTERVENTION ACTIONS

(71) Applicant: Ecovative LLC, Green Island, NY (US)

(72) Inventors: Eben D. Bayer, Troy, NY (US); Scott Elliot Baker, Albany, NY (US); Gavin Reim McIntyre, Troy, NY (US); Peter James Mueller, Poestenkill, NY (US); Jeff Andrew Mundt, Hackettstown, NJ (US); Julian Noah Rosenberg, Altamont, NY (US); Orkan Telhan, Cropseyville, NY (US); Jacob Michael Winiski, Troy, NY (US)

(21) Appl. No.: 18/964,042

(22) Filed: Nov. 29, 2024

Related U.S. Application Data
(60) Provisional application No. 63/550,271, filed on Feb. 6, 2024.
(60) Provisional application No. 63/604,763, filed on Nov. 30, 2023.

(51) Int. Cl. A01G 18/40; C12N 13/00

(52) U.S. Cl. CPC A01G 18/40; C12N 13/00

(57) ABSTRACT

Systems and methods for providing supplemental growth conditions to substrates, growth matrices, and growing aerial mycelium materials are provided which allow for enhanced aerial mycelial growth or targeted mycelial growth from either select or entire portions of substrates, growth matrices, or growing aerial mycelial materials (such as panels), or within select or entire portions of growth environments, when compared to growth levels and/or attributes resulting from base-line growth conditions. Such supplemental growth conditions may result from targeted application of specific chemistries, biological materials, or other materials applied to select or entire portions of substrates, growth matrices, growing aerial mycelium, or beds, trays, racks, webs, shelving units, growth environments containing such, or alternatively, from select physical manipulation or physical action steps imparted upon select or entire portions of substrates, growth matrices, growing aerial mycelium, or beds, trays, racks, webs, shelving units, growth environments containing such. In situ intervention of growing aerial mycelial tissue offers efficiencies and flexibility of material production and design, at earlier stages in a material lifecycle, compared to later stages for imparting similar design attributes. Such efficiencies and flexibility present distinct commercial advantages, translating into reduced resource consumption, reduced waste generation, and adaptable production design.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. In particular, this application claims filing benefit of U.S. Provisional Patent Application No. 63/604,763 having a filing date of Nov. 30, 2023 and U.S. Provisional Patent Application No. 63/550,271 having a filing date of Feb. 6, 2024, which are hereby incorporated by reference in their entirety.

### BACKGROUND

### Field

This application relates generally to improved methods and systems for growing aerial mycelium, and in particular, for altering growth and growth trajectories of aerial mycelium materials during their active vegetative growth phase within a growth environment, such as for example, either prior to, or after they have extended a desired distance from their solid-state fermentation growth matrix (e.g. inoculated particulate nutritive substrate).

### Background

The mycelium is the vegetative part of filamentous fungi, consisting of a network of fine filaments called hyphae. The principal role of a mycelium is to facilitate the growth and nutrient absorption of a fungus. The technological application of mycelia has recently gained significant interest due to their use as or in, e.g., biodegradable material, biofabrication, and environmental remediation.

One promising area of mycelium research has been the production of aerial mycelium, a form of mycelium having unique properties and potential applications, particularly in the food and textile industries. Aerial mycelia refer to hyphae that emerges from a colonized solid-state fermentation, particulate substrate, into an airmass directly adjacent to a mycelium contained within the particulate substrate. For a solid-state fermentation, particulate substrate (e.g., source of nutrition, growth substrate, or growth matrix), aerial hyphae under certain growth environment conditions, extend out and away from the substrate or matrix (and in some instances up to several or many inches away from a solid particulate substrate or matrix, with the substrate or matrix typically being a plant-based, particulate material), as opposed to extending solely in and immediately around the particulate substrate or matrix, between particles, or across or within a liquid surface. Such aerial hyphae can then be separated as a mass from the solid-state fermentation growth substrate or matrix as a discrete material, typically without significant additional steps needed to remove stray or embedded particulate substrate or matrix particulate material. For the purposes of this application, the solid-state growth substrate (as employed in solid-state fermentation processes), may be primarily a solid state or phase material, but may also encompass a slurry of solid state or phase particulates. Each of these states or phases should be distinguished from a liquid-state fermentation system (or submerged fermentation process) in which fungal nutrition is derived primarily from a liquid phase material, and the vast majority of the system is comprised of a continuous phase liquid, which is not self-supporting.

Given that the notion of aerial mycelium for commercial use is still in its relative infancy compared with other material production concepts, industrial production of aerial mycelia has required the creation of unique interior fungal growth spaces and methods to achieve consistent and efficient aerial mycelia growth on a commercial scale. Various contributing “base-line” growth conditions have thus far been identified for successfully generating robust aerial mycelium materials. These include temperature, humidity, mist levels (that is airborne liquid moisture particles), environmental gaseous content, airflow, light levels and type, and growth substrate/matrix nutritional content. Further, certain additional conditions or action steps have been identified to generate mycelium materials demonstrating desired attributes. For instance, the use of certain chemical and substance supplements, and certain environmental stimuli, have been associated with mycelium growth, or the growth of aerial mycelium demonstrating certain attributes. Such are described in United States Patent Publication 2015/0033620 to Greetham et al., directed to Mycological Biopolymers Grown in Void Space Tooling. Additional methods for stimulating the expression of specific tissue morphologies, albeit as part of a particulate composite, are described in U.S. Pat. No. 10,125,347 to Winiski. Furthermore, methods of producing a chitinous polymer derived from fungal mycelium in the form of fused sheets are described in U.S. Pat. No. 9,879,219 to McIntyre et al. Additionally, U.S. Pat. No. 10,687,482 to Ross et al., entitled Method of Producing Fungal Materials and Objects Made Therefrom, describes the use of means for directing the growth of mycelium.

However, even given the previously described conditions there is still a need for in situ environmental growth condition changes, physical and chemical interventions, and various synergistic combinations thereof, apart from the previously noted base-line environmental growth conditions, in order to amplify, adjust, or enhance growth levels of specifically-desired aerial mycelia materials, or to target specifically desirable aerial mycelium materials having particular end-product uses or desirable performance attributes. For example, in the textile space, there is still a need for in situ growth systems which, through physical manipulation of growing aerial mycelium, provide higher tensile strength materials for use in textile end products. In the food space, there are still needs for in situ growth methods and systems which eventually provide for more flavorful and desirably textured, aerial-mycelium based food products. That is, there is still a need for the identification and implementation of enhanced aerial mycelium growth methods and systems which result in the growth of consistent/predictable aerial mycelium materials having desired, pre-determined attributes, such as desirable tastes, textures, densities and other physical performance qualities not previously or sufficiently provided for.

### SUMMARY

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect of the invention, a method for providing supplemental growth conditions to inoculated substrates for the support of growing aerial mycelium materials, alternatively, to growth matrices for the support of growing aerial mycelium materials, or alternatively to growing aerial mycelium materials themselves, situated on either inoculated substrates or growth matrices, includes:
(a) providing a growth environment including at least an inoculated substrate for supporting the growth of aerial mycelium material, alternatively a growth matrix for supporting the growth of aerial mycelium material, or alternatively, growing aerial mycelium material, which growth environment supports base-line growth conditions for the growth of aerial mycelium material;
(b) conducting at least one of the following steps upon either of said inoculated substrate, growth matrix, or growing aerial mycelium material,
   (1) performing a physical (such as a mechanical) manipulation on at least one of the inoculated substrate, growth matrix, or growing aerial mycelium, such that the physical manipulation imparts a physical change in at least one of the inoculated substrate, growth matrix, or growing aerial mycelium, which physical change enhances at least one aerial mycelium material physical attribute when compared with aerial mycelium grown under the base-line growth conditions without the physical manipulation, with the physical change being selected from the group consisting of: selectively displacing substrate, growth matrix, or growing aerial mycelium, selectively depressing substrate, growth matrix, or growing aerial mycelium, selectively compressing substrate, growth matrix, or growing aerial mycelium, selectively layering growing aerial mycelium, selectively separating, texturing, or patterning portions of growing aerial mycelium, selectively aligning fibers of growing aerial mycelium via either direct contact or indirect contact techniques, and selectively creating spatial voids in the growing aerial mycelium,
   (2) adding a chemical, biologic, or other substance to at least one of the inoculated substrate, growth matrix, or growing aerial mycelium, such that the addition enhances at least one growing aerial mycelium attribute selected from the group consisting of improved taste as a result of the chemical, biologic, or substance incorporation or uptake from the addition into the structure of the growing aerial mycelium, or deposition on the growing aerial mycelium surface, desired color change as a result of the chemical, biologic, or substance incorporation or uptake from the addition into the structure of the growing aerial mycelium, or deposition on the growing aerial mycelium surface, desired improvement in nutritional content for human or animal consumption as a result of the chemical, biologic, or substance incorporation or uptake from the addition into the structure of the growing aerial mycelium, or deposition on the growing aerial mycelium surface, desired improvement in shelf-life of final aerial-mycelium based product as a result of the chemical, biologic, or substance incorporation or uptake from the addition into the structure of the growing aerial mycelium or deposition on the growing aerial mycelium surface,
(c) allowing growing aerial mycelium to continue growing in the growth environment in order to either develop an enhanced physical attribute selected from the group consisting of improved or targeted density, homogeneous or heterogeneous surface topology with limited or spatially-directed aerial mycelium bulbous occurrence per given unit area, or more aligned or patterned fiber orientation, or to develop an enhanced product performance attribute selected from improved taste, shelf-life, texture, mouth-feel, and nutritional content.

In another aspect of the invention, the grown aerial mycelium that has experienced either in situ physical manipulation or chemical, biologic, or substance addition to either a substrate, growth matrix, or growing aerial mycelium itself, is optionally terminated (in that the aerial mycelium organism is rendered inert, such that its biological metabolism has ceased) and/or separated from the substrate or growth matrix.

In another aspect of the invention, at least one physical manipulation is performed on the growing aerial mycelium in situ.

In another aspect of the invention, at least two physical manipulations are performed on the growing aerial mycelium in situ.

In still another aspect of the invention, at least one chemical, biologic, or substance addition is performed on either the inoculated substrate, growth matrix, or growing aerial mycelium in situ.

In yet another aspect of the invention, at least two chemical, biologic, or substance additions are performed on either the inoculated substrate, growth matrix, or growing aerial mycelium in situ.

In yet a further aspect of the invention, at least one physical manipulation and one chemical, biologic, or substance addition is performed on at least one of the inoculated substrate, growth matrix, or growing aerial mycelium in situ.

In yet a further aspect of the invention, at least one physical manipulation and one chemical, biologic, or substance addition is performed on at least one of the inoculated substrate, growth matrix, or growing aerial mycelium in situ concurrently in time, such that there is at least some overlap in time that such physical manipulation and chemical, biologic, or substance addition occurs.

In yet a further aspect of the invention, there is no overlap in time that at least one physical manipulation and one chemical, biologic, or substance addition is performed on at least one of the inoculated substrate, growth matrix, or growing aerial mycelium in situ.

In still another aspect of the invention, at least one physical manipulation and one chemical, biologic, or substance addition is performed sequentially on at least one of the inoculated substrate, growth matrix, or growing aerial mycelium in situ.

In yet another aspect of the invention, at least two physical manipulations are performed on at least one of the inoculated substrate, growth matrix, or growing aerial mycelium in situ, wherein the at least two physical manipulations are separated temporally (by a period of time) in which temporal gap there are either no physical manipulations occurring, or there are different physical manipulations occurring from those of the at least two physical manipulations.

In yet another aspect of the invention, two of the same physical manipulations are performed on at least one of the inoculated substrate, growth matrix, or growing aerial mycelium in situ, but such physical manipulations are separated temporally by a time period of no physical manipulation.

In yet another aspect of the invention, at least two chemical, biologic, or substance additions are performed on at least one of the inoculated substrate, growth matrix, or growing aerial mycelium in situ, wherein the at least two chemical, biologic, or substance additions are separated temporally (by a period of time) in which there are either no chemical, biologic, or substance additions occurring in the temporal gap, or there are different chemical, biologic, or substance additions occurring from those of the at least two chemical, biologic, or substance additions.

In yet another aspect of the invention, two of the same chemical, biologic, or substance additions are performed on at least one of the inoculated substrate, growth matrix, or growing aerial mycelium in situ, but such chemical, biologic, or substance additions are separated temporally by a time period in which there is no chemical, biologic, or substance addition.

In still a further aspect of the invention, temporal periods in which either physical manipulation or chemical, biologic, or substance addition occur, are separated by at least one temporal period in which either physical manipulation or chemical, biologic, or substance addition does not occur, and the growing aerial mycelium is left undisturbed to grow without supplemental physical manipulation or chemical, biologic, or substance addition.

In still a further aspect of the invention, the chemical, biologic, or substance addition in situ includes a material which imparts animal meat-like taste to the aerial mycelium.

In still a further aspect of the invention, the chemical, biologic, or substance addition in situ which imparts animal meat-like taste to the aerial mycelium reacts in the presence of a sugar and heat to form such taste.

In still a further aspect of the invention, the chemical, biologic, or substance addition in situ which imparts animal meat-like taste to the aerial mycelium is thiamine.

In yet another aspect of the invention, the physical manipulation in situ creates either a homogeneous or heterogeneous surface topology of the growing aerial mycelium.

In yet another aspect of the invention, the physical manipulation in situ is exposure to sound waves.

In yet another aspect of the invention, the physical manipulation is a direct contact with an apparatus in situ that either depresses, compresses, slices, cuts, scores, layers, abrades, scratches, creates voids, repositions portions of the aerial mycelium, or a combination thereof.

In still another aspect of the invention, at least one physical manipulation of the aerial mycelium in situ is accompanied by at least one chemical, biologic, or other substance addition in situ.

In yet another aspect of the invention, at least one physical manipulation is performed on the growing aerial mycelium in situ.

In yet another aspect of the invention, at least one chemical, biologic, or other substance addition is added to the growing aerial mycelium in situ.

In yet another aspect of the invention, at least one physical manipulation and/or at least one chemical, biologic, or other substance addition is performed in situ on the growing aerial mycelium, resulting in at least one enhanced aerial mycelium attribute selected from the group consisting of increased density, increased fiber alignment, targeted taste, targeted texture, increased surface homogeneity, increased surface heterogeneity, enhanced color, enhanced mouth feel, enhanced tear strength, enhanced tensile strength, enhanced elasticity, enhanced void space in the aerial mycelium (especially when compared with that achieved under base-line growth conditions without such supplemental condition change of either physical manipulation and/or at least one chemical, biologic, or other substance addition).

In still another aspect of the invention, a method for growing aerial mycelium (that is through solid state fermentation) includes first providing an inoculated solid-state fermentation substrate or growth matrix in a growth environment, secondly, establishing base-line growth conditions in the growth environment sufficient to encourage growth of aerial mycelium, thirdly, performing at least one of a physical/mechanical manipulation in situ to either the inoculated substrate, the growth matrix, or the growing aerial mycelium to alter or enhance the growth trajectory of the aerial mycelium, and then fourth, to optionally terminate and/or separate the growth of the aerial mycelium from the inoculated substrate or growth matrix.

In still another aspect of the invention, a method for growing aerial mycelium (that is through solid state fermentation) includes first providing an inoculated solid-state fermentation substrate or growth matrix in a growth environment, secondly, establishing base-line growth conditions in the growth environment sufficient to encourage growth of aerial mycelium, thirdly, performing at least one of a chemical, biologic, or other substance addition in situ to either the inoculated substrate, the growth matrix, or the growing aerial mycelium to alter or enhance the growth trajectory of the aerial mycelium, and then fourth, to optionally terminate and/or separate the growth of the aerial mycelium from the inoculated substrate or growth matrix.

In yet another aspect of the invention, a method for growing aerial mycelium through solid state fermentation includes the steps of first providing an inoculated substrate (comprised substantially of solid particulates or slurry containing solid particulates) or growth matrix in a growth environment for growing aerial mycelium, creating conditions within the growth environment sufficient to encourage the growth of aerial mycelium, performing at least one of a physical (or mechanical) manipulation in situ on either the inoculated substrate, growth matrix (that is the inoculated substrate), or growing aerial mycelium, allowing growing aerial mycelium to continue to grow within the growth environment, such that its continued growth exhibits an enhanced physical, structural, visual, or aesthetic attribute as a result of the at least one physical (or mechanical) manipulation.

In yet still another aspect of the invention, a method for growing aerial mycelium through solid state fermentation includes the steps of first providing an inoculated substrate (comprised substantially of solid particulates or slurry containing solid particulates) or growth matrix in a growth environment for growing aerial mycelium, creating conditions within the growth environment sufficient to encourage the growth of aerial mycelium, performing at least one of a chemical, biologic, or other substance addition in situ on either the inoculated substrate, growth matrix (that is the inoculated substrate with other materials), or growing aerial mycelium, allowing growing aerial mycelium to continue to grow within the growth environment, such that its continued growth exhibits an enhanced physical (such as including textural change, taste change, or mouth-feel enhancement), structural, visual, nutritional or shelf-stability, or aesthetic attribute as a result of the at least one chemical, biologic, or other substance addition.

In yet another aspect of the invention, a composition of aerial mycelium composition is disclosed. The composition comprises an aerial mycelium and a chemical agent, a biologic agent, or other substance. The chemical agent, biologic agent, or other substance is inert or relatively inert with respect to the fungal organism's metabolic activity and which upon exposure to processing treatment reflects at least one enhanced aerial mycelium attribute selected from the group consisting of increased density, increased fiber alignment, targeted taste, targeted texture, increased surface homogeneity, increased surface heterogeneity, enhanced color, enhanced mouth feel, enhanced tear strength, enhanced tensile strength, enhanced elasticity and enhanced void space in the aerial mycelium (especially when compared with that achieved under base-line growth conditions without such at least one chemical, biologic, or other substance addition).

In yet still another aspect of the invention, a composition of aerial mycelium composition is disclosed. The composition comprises a growing or grown aerial mycelium, said aerial mycelium including mechanically-induced, mycelial tissue damage which causes a change in coloration of the mycelial tissue making up the growing or grown aerial mycelium. Said change in coloration of the mycelial tissue is independent of any secondary coloring agents, such as dyes or pigments.

In another aspect of the invention, said mechanically-induced mycelial tissue damage has been selectively placed in discrete locations along at least one dimension of the growing or grown aerial mycelium.

In yet another aspect of the invention, a composition of aerial mycelium composition is disclosed. The composition comprises a growing or grown aerial mycelium, said growing or grown aerial mycelium including within its tissue either a deposited chemical agent, biologic agent, or other additive substance. The deposited chemical agent, biologic agent, or other additive substance causes a change in condition of the mycelial tissue making up the growing or grown aerial mycelium, upon later activation through either a chemical reaction, process step, or change in environmental condition.

In another aspect of the invention, said deposited chemical agent, biologic agent, or other additive substance causes a change in either the texture, color, flavor, aroma, or strength of said mycelial tissue upon later activation through either a chemical reaction, process step, or change in environmental condition.

In another aspect of the invention, said deposited chemical agent, biologic agent, or other additive substance causes a change in flavor of said mycelial tissue, upon later activation through a process step.

In another aspect of the invention, said deposited chemical agent causes a change in flavor of said mycelial tissue, upon later activation through a heating process step.

All of these embodiments are intended to be within the scope of the invention herein disclosed. These and other embodiments of the present invention will become readily apparent to those skilled in the art from the following detailed description of the alternative embodiments having reference to the attached figures, the invention not being limited to any particular alternative embodiment(s) disclosed.

### BRIEF DESCRIPTION OF THE DRAWINGS

The features and advantages of the methods and systems described herein will become more fully apparent from the following description and appended claims, taken in conjunction with the accompanying drawings. These drawings depict only several embodiments in accordance with the disclosure and are not to be considered limiting of their scope. In the drawings, similar reference numbers or symbols typically identify similar components, unless context dictates otherwise. In some instances, the drawings may not be drawn to scale.

FIG. 1A illustrates an embodiment of a growth matrix (that is an inoculated substrate with other materials) in a first exemplary growth environment, suitable to support or encourage extra-particle aerial mycelial growth and establishing base-line growth conditions.

FIG. 1B illustrates an embodiment of extra-particle aerial mycelial growth extending from the growth matrix of FIG. 1A in said growth environment.

FIG. 2A illustrates an additional exemplary embodiment of a growth environment (growth chamber) with multiple shelves of trays growing aerial mycelium.

FIG. 2B illustrates a third exemplary embodiment of a portion of a growth environment (growth chamber) with multiple shelves for trays, beds, racks, webs, or nets for growing aerial mycelium.

FIG. 3 illustrates through a flow diagram, a base-line method for growing aerial mycelium through solid state fermentation under base-line growth conditions, in a growth environment.

FIG. 4 illustrates through a flow diagram, one method for growing aerial mycelium through solid state fermentation under base-line growth conditions in a growth environment, but also with the additional assistance of a supplemental condition, that is a physical or mechanical manipulation in situ to either the growing aerial mycelium, growth matrix, or inoculated substrate in order to alter or enhance the growth trajectory of the growing aerial mycelium.

FIG. 5 illustrates through a flow diagram, another method for growing aerial mycelium through solid state fermentation under base-line growth conditions, in a growth environment, but also with the assistance of a supplemental condition, that is a chemical, biologic, or other substance addition in situ to either growing aerial mycelium, growth matrix, or inoculated substrate in order to alter or enhance the growth trajectory of the growing aerial mycelium.

FIG. 6 illustrates, through a flow diagram, another method for growing aerial mycelium through solid state fermentation under base-line growth conditions, in a growth environment, but also with the assistance of supplemental in situ conditions, that are: an optional combination of one or more physical or mechanical manipulations and/or one or more optional chemical, biologic, or other substance additions to either the growing aerial mycelium, growth matrix, or inoculated substrate in order to enhance the growth trajectory of the growing aerial mycelium.

### DETAILED DESCRIPTION

U.S. Pat. No. 11,277,979 to Greetham et al., International PCT Patent Application No. WO2019/099474A1 to Kaplan-Bic et al., PCT Patent Application No. WO2023172696 to Snyder et al., PCT Patent Application No. WO2022235688 to Winiski et al., and PCT Patent Application No. WO2022235694 to Carlton et al., the entirety of each of which are incorporated herein by reference thereto except where inconsistent with the disclosure herein, describe methods of growing a mycological biopolymer material, aerial mycelium and products resulting therefrom, including environmental conditions in which such aerial mycelium materials may be grown. The methods of the current disclosure may utilize environmental conditions as described in the preceding references, to provide base-line growth conditions for aerial mycelium growth methods and systems.

Described herein are embodiments of modified systems, modified methods, and apparatus to consistently grow enhanced and/or targeted (altered) aerial mycelium materials which growth trajectory is changed, as a result of supplemental conditions imposed on either the inoculated substrate, growth matrix, or growing aerial mycelium in situ. Essentially, the aerial mycelia grown through a solid-state fermentation particulate process, experiences a type of in-process intervention, which alters the finally grown aerial mycelium material. The enhanced or targeted aerial mycelium material that is grown via such systems can be used in the food industry (for example, as an animal-based, meat-substitute product, and one that may present to the consumer a product that offers the appearance and texture of traditional animal-based meat material (e.g., beef, pork, poultry and seafood), and in other industries, such as textiles, packaging, and others.

It is a further object of the present invention to provide mycelial growth methods that offer flexibility such that the methods are capable of toggling back and forth between various mycelial growth step options (among several available, such as in sequence of steps, or overall steps utilized), or alternatively that are adaptable to the differing requirements of various mycelial organism strains as to accommodate either varying product configurations (with each design demonstrating differing desired end-product attributes), or to accommodate various manufacturing/growth facility spaces or growth equipment availability.

It is a further object of the present invention to provide methods and systems for growing enhanced or targeted aerial mycelium that are consistent, repeatable, adaptable to various technology platforms, and energy efficient, while providing for predictably high quality and quantity mycelium-based materials that are useful, practical, and which may be produced at any scale, from small-scale benchtop biological reactors to large-scale manufacturing facilities. The enhanced, altered, or targeted aerial mycelium can comprise, consist essentially of, or consist of fungal aerial mycelium. The enhanced, altered, or targeted aerial mycelium can comprise, consist essentially of, or consist of mature aerial mycelium. The enhanced, altered, or targeted aerial mycelium may comprise multiple types of enhanced, altered, or targeted aerial mycelium in a single aerial mycelium panel, depending upon relative exposure of portions of the growing aerial mycelium to supplemental growth conditions, or various in situ process steps. Growth environments may include multiple types of enhanced, altered, or targeted aerial mycelium in the same growth environment, depending upon relative exposure of portions of the growing aerial mycelium in the growth environment.

It is another object of the present invention to provide methods and systems of growing enhanced or targeted aerial mycelium that comprise at least aerial mycelium grown from a solid-state fermentation process.

### DEFINITIONS

“Mycelium” as used herein refers to a connective network of fungal hyphae, with mycelia being the plural form of mycelium.

“Hyphae” or “hypha” as used herein refers to branched filament vegetative cellular structures that are interwoven to form mycelium.

“Fruiting body” as used herein refers to a fungal stipe, pileus, gill, pore structure, or a combination thereof, and may be referred to herein as “mushroom.”

“Substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an aerial mycelium of the present disclosure. Suitable substrates are disclosed, for example, in United States Publication 20200239830A1 to O'Brien et al., the entire contents of which are hereby incorporated by reference in their entirety to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US20200239830A1, and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets and combinations thereof. Additional useful substrates for the growth of mycelia are disclosed herein. A substrate can also be a depleted substrate, which is at least partially depleted of nutrients or other materials after extra-particle aerial mycelial growth has been grown and divided from the growth matrix to form a separated aerial mycelium. A substrate or a depleted substrate can be a substrate which has been further processed (e.g., chemically or mechanically) to improve its viability to support new mycelial growth (e.g., extra-particle aerial mycelial growth).

“Growth media” or “growth medium” as used herein refers to a matrix containing a substrate and an optional further source of nutrition that is the same or different than the substrate, wherein the substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Inoculated substrate” as used herein refers to a substrate that has been inoculated with fungal inoculum. For example, an inoculated substrate can be formed by combining an uninoculated substrate with a fungal inoculum. Alternatively, an inoculum can be a solid or liquid composition of any living organism or part thereof, including, but not limited to, bacteria, archaea, viruses, protozoa, algae, animal cells or tissue, plant cells or tissue, or any other living material.” An inoculated substrate can be formed by combining an uninoculated substrate with a previously inoculated substrate. An inoculated substrate can be formed by combining an inoculated substrate with a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal species, asexual sporulation occurs as part of normal vegetative growth, and as such could occur during the colonization process. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing aqueous mist into the growth environment and/or depositing aqueous mist onto colonized substrate and/or any ensuing extra-particle growth. In some embodiments, the use of aqueous mist can be adjusted, for example, to desired levels, direction, composition, and timing, to affect the topology, morphology, density, and/or volume of the growth. In some further embodiments, mist can be comprised of two or more liquid compositions. For example, introduction of liquid mist can be sourced from reservoirs of liquid water, liquid nutrients, liquid dye, liquid flavoring, liquid texturizing solutions, liquid tenderizing solutions, liquid mineral solutions, or any other liquid solution that can affect the topology, geometry and/or morphology of aerial mycelium.

Any suitable solid-state fermentation, particulate substrate can be used alone, or optionally combined with a nutrient source, as media to support mycelial growth. The growth media can be hydrated to a final target moisture content prior to inoculation with a fungal inoculum. Growth media hydration can be achieved via the addition of any suitable source of moisture. In a non-limiting example, the moisture source can be airborne or non-airborne liquid phase water (or other liquids), an aqueous solution containing one or more additives (including but not limited to a nutrient source), and/or gas phase water (or other compound). In some embodiments, at least a portion of the moisture is derived from steam utilized during bioburden reduction of the growth media. In some embodiments, inoculation of the growth media with the fungal inoculum can include a further hydration step to achieve a target moisture content, which can be the same or different than the moisture content of the growth media. For example, if growth media loses moisture during fungal inoculation, the fungal inoculated growth media can be hydrated to compensate for the lost moisture.

Methods for the production of aerial mycelium disclosed herein can include an inoculation stage, wherein an inoculum is used to transport an organism into a substrate. The inoculum, which carries a desired fungal strain, is produced in sufficient quantities to inoculate a target quantity of substrate. The inoculation can provide a plurality of myceliation sites (nucleation points) distributed throughout the substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and at least one fungus. The inoculum may contain enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary spawn types provided by inoculum retailers. In some aspects, a fungal inoculum can be characterized by its density. In some embodiments, a fungal inoculum has a density of about 0.1 gram per cubic inch to about 10 grams per cubic inch, or from about 1 gram per cubic inch to about 7 grams per cubic inch. A skilled person can modify variables including the substrate or growth media component identities, substrate or growth media nutrition profile, substrate or growth media moisture content, substrate or growth media bioburden, inoculation rate, and inoculum constituent concentrations to arrive at a suitable medium to support aerial mycelial growth.

“Growth environment” as used herein refers to an environment that supports the growth of mycelia, as would be readily understood by a person of ordinary skill in the art in the mycelial cultivation industry, which contains a growth atmosphere having a gaseous environment of carbon dioxide (CO₂), oxygen (O₂), and a balance of other atmospheric gases including nitrogen (N₂), and which is further characterized as having a relative humidity. In some aspects of the present disclosure, the growth atmosphere can have a CO₂ content of at least about 0.02% (v/v), at least about 0.6%, at least about 5% (v/v), less than about 10% (v/v), less than about 8% (v/v), less than about 7%, between about 0.02% and 10%, between about 0.02% and 8%, between about 0.6% and about 7%, between about 5% and about 10%, or between about 5% and about 8%. In some other aspects, the growth atmosphere can have an 02 content of at least about 12% (v/v), or at least about 14% (v/v), and at most about 21% (v/v). In yet other aspects, the growth atmosphere can have an N2 content of at most about 79% (v/v). Each foregoing CO₂, O₂ or N₂ content is based on a dry gaseous environment, notwithstanding the growth environment atmosphere relative humidity. “A portion of the growth environment” as used herein refers to a percentage of the total volume of the growth environment. For example, a portion of the growth environment can encompass between 0.01% to 100% of the total volume of the growth environment. A portion of the growth environment can refer to any fraction of the one-dimensional, two-dimensional or three-dimensional geometry comprising the growth environment. For example, a portion of the growth environment can refer to the unit length, the unit width, the unit height, the unit body diagonal, the unit face diagonal, the unit perimeter, the unit radius, the unit circumference, the unit surface area, the unit cross section, or the unit volume of the growth environment.

The geometry of the growth environment can be customized to support mycelium growth at several spatial scales. In some embodiments, the volume of the growth environment can fall within a range of between about at least 0.1 ft³ and/or less than or equal to about 500,000 ft^{3,} or can fall within a range between about at least 1.0 ft³ and/or less than or equal to 250,000 ft³. In some yet further embodiments, the volume of the growth environment can be about 0.1 ft³, 0.2 ft3, 0.3 ft³, 0.4 ft³, 0.5 ft³, 0.6 ft³, 0.7 ft³, 0.8 ft³, 0.9 ft³, 1.0 ft³, or any range therebetween. In some yet further embodiments, the volume of the growth environment can be about 250,000 ft³, 300,000 ft³, 400,000 ft³, 500,000 ft³, or any range therebetween.

A growth environment can comprise one or more sub-environments. For example, each sub-environment can be comprised of aerial mycelium at different growth stages.

“Aerial mycelium” as used herein refers to mycelium obtained from extra-particle aerial mycelial growth, and which is substantially free of growth matrix (and is that part of mycelial growth that extends away from and apart from a solid-state fermentation particulate inoculated substrate or growth matrix).

“Mature mycelium” as used herein refers to mycelium that is still in contact with the solid-state fermentation, particulate growth medium, growth media, or inoculated substrate and is suitable for use. In some embodiments, the mature mycelium can be mature aerial mycelium.

“Extra-particle mycelial growth” (EPM) as used herein refers to mycelial growth, which can be either appressed or aerial.

“Extra-particle aerial mycelial growth” as used herein refers to a distinct mycelial growth that occurs away from and outward from the surface of a growth matrix. Extra-particle aerial mycelial growth can exhibit negative gravitropism. In a geometrically unrestricted scenario, extra-particle aerial mycelial growth could be described as being positively gravitropic, or neutrally gravitropic, aerial, and radial in which growth will expand in all directions from its point source. In some embodiments, external forces, such as airflow, can be applied towards (e.g., approximately perpendicular to the growth environment floor) the growth substrate, and in some embodiments, through the growth substrate, for example, to create downward aerial mycelium growth in the direction of gravity. Alternatively, airflow can be applied across the growth substrate in a manner parallel or horizontal to the growth substrate surface.

“Positive gravitropism” as used herein refers to growth that preferentially occurs in the direction of gravity.

“Negative gravitropism” as used herein refers to mycelial growth that preferentially occurs in the direction away from gravity. As disclosed herein, extra-particle aerial mycelial growth can exhibit in one embodiment negative gravitropism. Without being bound by any particular theory, this may be attributable at least in part to the geometric restriction of the growth format, wherein an uncovered tool having a bottom and side walls contains a growth matrix. With such geometric restriction, growth will primarily occur along the unrestricted dimension(s), which in the scenario is primarily vertically (negatively gravitropic).

“Growth run” or “run” as used herein refers to the time period under specific environmental conditions during which a mature mycelium is formed. In some embodiments, a growth run or run can be synonymous with or comprise of incubating. Aerial mycelia of the present disclosure can be grown in a matter of weeks or days. In some embodiments, a growth run is of a duration between about 10 days and 164 days, alternatively between about 10 days and 14 days. This feature is of practical value in the production of food ingredients or food products, where time and efficiency are at a premium. Accordingly, the presently disclosed method of making an aerial mycelium comprises incubating a growth matrix in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 to about 13 days, or within a range of about 7 to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

Advantageously, incubating a growth matrix comprising a colonized substrate (wherein said colonized substrate comprises a growth medium previously colonized with mycelium of a fungus) in a growth environment of the present disclosure can result in earlier expression of aerial mycelial tissue compared to incubation of a growth matrix comprising substantially the same or a similar growth medium and a fungal inoculum, wherein the fungal inoculum contains a fungus. Accordingly, a method of making an aerial mycelium of the present disclosure can comprise incubating a growth matrix comprising a colonized substrate (wherein said colonized substrate comprises a growth medium previously colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, aerial mycelia of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

“Mycelium-based” as used herein refers to a composition substantially comprising mycelium. “Aerial-mycelium based” as used herein refers to a composition substantially comprising aerial mycelium.

“Homogeneous” as used herein refers to the topology of growth of the aerial mycelium. In some embodiments, aerial mycelium is morphologically composed of variably expressed structures (e.g., bulbous structures) with varying degrees of diffusion within and between one another, and in height, with respect to each other. This may be referred to more generally as the “topology of growth,” “growth topology” or “surface topology.” The variable and eccentric expression of bulbous features and variable tissue density within and between bulbous features represents a challenge for example, in textiles applications. For example, tensile failure can selectively occur when morphological “bulb” forms become too discrete, due to a lack of cross-linking at the intersections between these forms, which can lead to variable failure modes and reduced physical strength. Conversely, increased homogeneity can increase tensile strength, for example, by increasing cross-linking.

“In situ” as used herein, refers to a process or method step that occurs within a controlled, aerial mycelium, growth environment, such as for example, either to an inoculated substrate or inoculated substrates, to a growth matrix or growth matrices, or to one or more growing aerial mycelium, each within a controlled, aerial mycelium, growth environment.

“Lifecycle” as used herein refers to the developmental stages that fungi undergo, encompassing both sexual and asexual reproduction. For example, this process can involve distinct phases such as spore germination, hyphal extension, mycelial vegetative growth, formation of aerial mycelium/a, and/or the formation of specialized structures for reproduction.

“Solid-state fermentation” or “solid-state substrate fermentation” as used herein refers to a process wherein one or more organisms are grown on a solid substrate. For example, this process can involve a mycelium growing between and/or within the empty spaces of the solid growth matrix or substrate, thereby providing a solid physical support for growth. As noted, this method of fungal fermentation typically employs plant-based or inorganic-particle based materials that are either present in a solid phase or slurry phase.

“Base-line growth conditions”, as used in this application, refers to those environmental growth conditions which achieve a certain level of aerial mycelium growth. Such base-line growth conditions include temperature level(s), humidity level(s), gaseous content/level(s), mist level(s) and/or rates of application rates, airflow level(s) or application rates, and at times or periods, light types/levels, each condition depending on species of fungal organism for which aerial mycelium growth is desired. Examples of base-line growth conditions are enumerated in patent references incorporated by reference herein.

“Supplemental growth conditions”, as used in this application and which are specifically enumerated below, refers to additional environmental growth conditions, or action steps, distinct from base-line growth conditions and in most instances, in addition thereto, which are implemented in a growth environment (such as a BTBR or growth chamber or room) during an aerial mycelial growth phase, to produce more enhanced, altered, or targeted aerial mycelial growth, when compared with that growth experienced from base-line growth conditions, alternatively, distinguishable aerial mycelial growth when compared with that experienced from base-line growth conditions, or a targeted aerial mycelial growth (such as one that is distinguishable from aerial mycelial growth experienced under base-line growth conditions). Such distinguishable aerial mycelial growth is distinguishable from the aerial mycelial growth which would otherwise grow under the base-line growth conditions for the fungal organism, were such additional environmental growth conditions or action steps not to be taken and include changed trajectories of growth as a result of the additional conditions or action steps. The change in aerial mycelial growth is at least in part attributable to the implemented supplemental condition. As an example of supplemental growth conditions, the addition of one or more specifically enumerated chemical, biologic, or other additional substance treatments to an inoculated substrate, growth matrix, or growing aerial mycelium (such as for example only, to a particular surface directly exposed to a growth environment atmosphere) in a growth environment that otherwise would not have been added to such an inoculated substrate, growth matrix or growing aerial mycelium, may alter the subsequent aerial mycelium material grown, to present as one that includes a particular taste, mouth-feel, surface texture to the touch, visual appearance, improved shelf-life, tensile strength, density, and tear strength as compared to an aerial mycelium material grown merely under the base-line growth conditions without such supplemental addition of chemical treatment.

It should be understood that the addition of a supplemental chemical, biologic, or substance treatment or physical (or mechanical) manipulation action taken, may be implemented through one or more various mechanisms, depending on the treatment. Additionally, the application of the supplemental conditions (whether it be supplemental chemical, biologic, or substance addition(s), or physical (or mechanical) manipulation(s) may be periodic or repeated throughout an aerial mycelium growth run. As examples, a chemical or biologic treatment may be applied through a periodic or continuous mist application or may alternatively be applied through introduction via a periodic or continuous drip or other dispenser at the base of the inoculated substrate or growth matrix for instance. Such a chemical or biologic treatment (or other substance addition, such as a particular mineral addition) may include one that either directly alters the growth of the aerial mycelium or indirectly changes the growing aerial mycelium material, such as by being taken up into or absorbed into the organism structure to change the organism anatomy or expressed phenotypical features, or is affixed to a grown aerial mycelium material exposed surface(s). Such chemical, biologic, or other substance introductions may be made to the inoculated substrate, growth matrix or growing aerial mycelium materials during various times in the lifecycle, and may include materials described for instance (for improved nutritive content) in U.S. Pat. No. 9,700,067 to Fraser et al. A supplemental growth condition may also include a physical or mechanical manipulation of, or physical action taken upon an inoculated substrate, growth matrix, or growing aerial mycelium in a growth environment, such as for example, a scoring, slicing, cutting, depression, or compression action of the growing aerial mycelium or substrate or growth matrix (in either discrete locations or homogeneously across the entire growing aerial mycelium, inoculated substrate, or growth matrix). Such an action may also result in the creation of void spaces in the growing aerial mycelium materials. Such an action may also expose the growing aerial mycelium to supplemental environmental stimuli such as electric fields or energy fields in situ. Additional physical manipulation actions may include the dragging of additional materials across the actively growing aerial mycelial tissue in situ, otherwise impacting the growing aerial mycelial tissue, and blowing (continuously or pulsed) the growing aerial mycelial tissue with directed jets (from one or multiple directions, with such jets including or not including additional chemistries or gas components). Still further environmental stimuli, which for the purposes of this application are considered physical manipulation, include exposure to sound and light waves.

In a further alternative, a combination of physical manipulations may be implemented upon the inoculated substrate, growth matrix, and/or growing aerial mycelium in order to accomplish a particular growth objective. For instance, growing aerial mycelium may be exposed to a cutting process wherein a portion of the aerial mycelium is in part, cut away from the inoculated substrate. The cut-away aerial mycelium may then be folded upon itself, optionally rolled, and permitted to regrow, or continue growing. In such a fashion, the final grown aerial mycelium will demonstrate several features (such as for instance density gradients, differentiated surfaces, or spatial voids) directly attributable to the string of supplemental physical manipulations.

Such a physical action may be along one or more axis of the inoculated substrate, growth matrix, or growing aerial mycelium in the growth environment, and be implemented to produce features like altered, final material physical topography when compared with material grown just under base-line growth conditions. Such resulting final physical features may include the targeted placement of pronounced and visually-apparent surface protrusions or other enhanced growth attributes in discrete locations or throughout the final grown aerial mycelium panel, or their reduction in occurrence, or elimination in their entirety such as for example resulting in altered density, or increased appearances of cellular void spaces within the final grown aerial mycelium material.

### DETAILED DESCRIPTION OF FIGURES AND EXAMPLES

The described invention provides for enhanced aerial mycelial growth or targeted aerial mycelial growth, compared to aerial mycelial growth resulting from merely base-line growth conditions. The enhanced or targeted aerial mycelial growth demonstrates differences in either physical or chemical attributes (such as structural/cellular differences), as a result of exposure to supplemental environmental growth conditions that differ from those resulting merely from base-line growth conditions, typically said supplemental environmental growth conditions being in addition to base-line growth condition exposure, or a result of variations in certain base-line growth conditions. Essentially, the supplemental growth conditions are imparted directly or indirectly to either select portions of, or the entirety of inoculated substrate(s), growth matrix(ces), or growing aerial mycelium materials in growth environments (in situ within the growth environments), or select portions of, or the entirety of the growth environments, such as select portions of the atmosphere (such as void space) contained within the growth environment. The supplemental growth conditions include physical manipulations (or physical action steps), typically resulting from mechanical apparatus/devices which act on the materials, and/or introduction of chemical, biologic, or other substance addition to the materials (or portions thereof), or to the growth environment (or portions thereof). By applying such physical action steps and/or substance introduction at select times and/or locations within the growth/lifecycle of the fungal organism, the fungal aerial mycelium growth patterns, growth trajectory, or structure may be altered to be more suitable with select product end uses, and the associated desired end product physical attributes. The in situ growth intervention methods and systems, provide flexibility in producing a variety of products from the same starting material, or from differing starting materials, using the same growth environment. Such flexibility of growth operation allows for efficient use of resources (such as substrate (inoculated or otherwise), growth matrices, energy, nutrients, water etc.), with reduced waste of grown aerial mycelium product. Essentially, end products can be grown on demand, demonstrating variations of desired attributes in accordance with evolving customer or consumer needs.

For instance, it has been found that certain mechanical properties of aerial mycelium-based products derived substantially from aerial mycelium may be significantly dependent on aerial mycelium densities, which may require meaningfully exceeding the base-line growth density, normally seen of aerial mycelium grown under base-line conditions. As an example, certain food or textile products may benefit from certain densities. By implementing one or more supplemental physical manipulation steps during aerial mycelial growth, resulting growth density levels may be increased above values realized under just base-line growth conditions.

In addition to densification, design motifs, aesthetic manipulations, material homogeneity attributes, fiber alignment, and textures may also be imparted to growing aerial mycelium products in situ during the growth phase, within the growth environment. Utilizing in situ interventions during the growth process of aerial mycelium for densification and/or imparting design or aesthetic features, allows for multiple advantages, including: (1) more efficient manufacturing methodologies by reducing the need for further manufacturing steps, and (2) continued or regrowth of aerial mycelium material after or between physical manipulations or substance introduction (or a combination thereof), allowing for progressive densification or accumulation of layered design or aesthetic features, or progressive deposition of chemicals, biologics, or other substance additions in layers (or accumulation within the organism tissue) through the thickness of the growing or grown aerial mycelium. As an example, “watermarking” type aesthetic effects may be imparted in a progressive series of steps.

Additionally, it is recognized that fungal tissue demonstrates specific responses to physical damage and manipulation, including increased branching and anastomosis at the damage site or which is distributed throughout the thallus, or such as through metabolic investment in greater frequencies of skeletal hyphae growth. In this case, physical interjections in the growth process, allowing for these biological responses, provide opportunities for increasing crosslinking and thallic durability leading to increased mechanical strength or material toughness, either throughout an entire tissue structure, or at desired target locations. These advantages may be considered independently from tissue densification where physical damage without densification may result in cross-linking and branching effects (such as simulating the effect of foraging insects on mycelium generally).

Additionally, fungal tissue demonstrates bruising, guttation, extra-cellular mucilage production, and pigmentation in response to physical damage or manipulation. In this case, interjection of physical actions, or manipulations during the in situ growth process leverages these responses for both aesthetic or mechanical purposes, such as to result in desired color changes and physical attribute changes. For example, a bruising or pigmentation response may be used to impart aesthetic designs, both in color and shape (or a combination thereof), or the development of extra-cellular mucilage high in α-glucans or polysaccharides which can increase stiffness levels of an aerial mycelium material or portions thereof, or further, to be leveraged as a heat-set binder.

Still further, for food end-product applications specifically, physical manipulation and the accompanying biological responses described above provide an opportunity for designing specific textural motifs, for example, simulating meat and fat density distributions associated with whole cut meats. This can result in specific mouth-feels and/or chewing sensations.

In one embodiment, the primary focus of physical manipulations or action steps can be a periodic, physical (in situ) disturbance of the growing hyphae to achieve uniformity throughout all panel dimensions, such as by slicing, cutting, piercing, scoring, abrading, puncturing or otherwise disrupting one or more fungal bulbs, compressing one or more individual fungal bulbs (ether in select locations or across the entire panel dimension), repositioning, or reorienting the growing aerial mycelial fungal tissue, dragging or rolling, needling, cutting and repositioning or other direct or indirect contact manipulations. Such indirect manipulations may include exposure of growing aerial mycelium material to light or sound waves (or various energy forms) at one or more portions or during one or more temporal periods of a growth run. The manipulations may substantively alter the growing aerial mycelium fiber orientation or agglomeration of the growing aerial mycelium which may be beneficial to a desired end product that will utilize the grown aerial mycelium material. These contacts demonstrate various approaches to homogenize panel morphology (reduce bulb appearances), impact fiber alignment, and/or densify the material. Of course, the physical manipulations may also be used to encourage the appearance of certain physical features, aside from increasing the relative percentage of relatively smoother surface topology between bulbous forms.

Also, by application of specific chemistries, biologic materials, or other substances, into or onto inoculated substrates, growth matrices, or the growing aerial mycelium (such as for example on surfaces thereof in the form of mist or graduated drips), or portions thereof, substances can be ingested by the fungal organism and either incorporated within the resulting aerial mycelial structure unchanged, or contribute to the nutritional content of the finally grown aerial mycelium product. Such substances may also be further manipulated through later applied processes, such as heating or pasteurization, which can change the added chemistry to provide for more meat-like attributes. For example, chemistry added as potential flavor enhancers, may be modified through the introduction of heat from pasteurization, to resemble chemistry found in meat, providing meat-like flavors. Such chemistries or substances may be added to either the inoculated substrate, growth matrix, or growing aerial mycelium over the period of growth within the growth environment. Thiamine might be one example of such a substance. Other substances may include, but are not limited to, garlic, onion, and/or various active compounds thereof. For example, one active compound may include allicin. Other chemistries, agents, or substantives may include, but are not limited to, alcohols (e.g., 2-butanol, ethanol, isobutanol, etc.), aromatic compounds (e.g., vanillin, etc.), lactones (e.g., decalactone, butyrolactone, etc.), esters (e.g., alkyl acetates, alkyl butyrates, alkyl butanoates, alkyl propionates, etc.), pyrazines, ketones (e.g., acetophenone, acetone, butanone, octanone, etc.), fatty acids (e.g., acetate, butyrate, caproate, isobutyrate, etc.), aldehydes (e.g., acetaldehyde, etc.), etc. In one embodiment, the selected chemistry may not negatively impact growth or metabolic activity of the organism utilized in creating the mycelium.

Chemistries, agents, or other substances which directly express attributes in the growing or grown mycelial tissue, either upon absorption or coating by/of the mycelia during growth (either through metabolic up-take in the normal course, such as via the provided nutrition or water vehicle in the substrate, or via direct application to the growing tissue, such as through mist applied to the growing tissue), or following up-take or contact, and upon later activation through a later process step acting on the growing or grown mycelia. Such chemistries may immediately react or otherwise be expressed directly in the growing or grown aerial mycelium tissue, such as coloring agents which are immediately visible (either through addition of substances, or through creation in response to a later reaction or trigger, or alternatively, via damage to the mycelial tissue by a mechanical physical manipulation) that do not negatively impact the normal fungal metabolism, and in one embodiment, do not pose any safety concerns to consumers, either through ingestion or surface contact by consumers. In some embodiments, they may become evident or visible at a later time. In one embodiment, such color-providing agents or color-initiating actions (such as simply in response to a mechanically induced stress or tissue damage) do not, in one embodiment, utilize traditional dye or pigment chemistries, although in a further embodiment, they may. It is envisioned that the substances may in one embodiment, be inert with respect to fungal metabolism, and then upon some later secondary trigger, whether it be by later chemical reaction, or change in mycelial condition, such as through environmental or process step exposure to heat, light wavelength, drying, boiling, or other cooking step for instance, or other physical action, such as a certain level of tissue agitation, the substances either react to produce a second chemistry (such as an expressed flavorant or aroma), or are otherwise allowed to express themselves in a finished product utilizing the aerial mycelial tissue.

Chemicals or other substances may also be applied as coatings to the growing aerial mycelium, which would be less likely to be absorbed or ingested within the fungal organism. Such coatings or substances may offer additional aesthetic or functional benefits to the final grown material.

Furthermore, chemicals may be applied to the growing aerial mycelium that may eventually result in one or more further reactions with either the molecular structure of the aerial mycelium or substances produced by the aerial mycelium during growth, which reactions will result in more desirable end products. For instance, in the context of textiles, if applied mist were to include sulfur salts, such chemistry may induce disulphide bonding to toughen the material between hydroxyls on the chitin and glucan chains of the fungal structure. In a further alternative, a mineral may be deposited that assists with ionic cross-linking. In the context of food, a substance may be added to either the inoculated substrate, growth matrix or growing aerial mycelium, which when processed by the fungal inoculum during aerial mycelium growth, reacts to become a more appealing chemistry to the consumer of such food.

The supplemental conditions including mechanical actions or additives described, may be implemented in multiple numbers, and in multiple combinations, such as including both mechanical and additive applications. Such supplemental conditions may be spaced apart temporally during their application or may be applied during the same time frame.

The figures and the following description relate to various example embodiments by way of illustration only. It should be noted that from the following discussion, example embodiments of the structures, methods, and systems disclosed herein will be readily recognized as viable alternatives that may be employed without departing from the principles of what is claimed. Reference will now be made in detail to several embodiments, examples of which are illustrated in the accompanying figures. It is noted that wherever practicable, similar or like reference numbers may be used in the figures and may indicate similar or like functionality. The figures depict embodiments of the disclosed methods or systems (with select equipment for purposes of illustration) and one skilled in the art will readily appreciate from the following description that various example embodiments of the structures, systems, methods and equipment described herein may be employed without departing from the principles described herein.

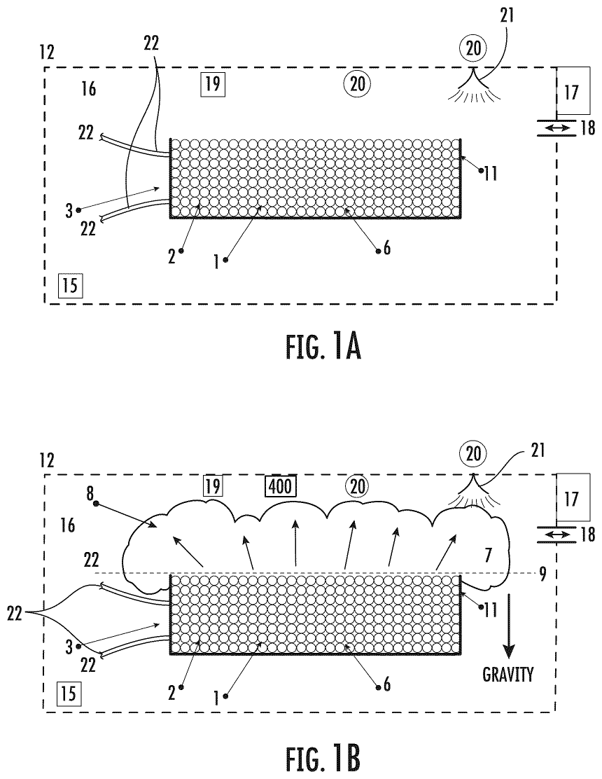

FIG. 1A illustrates an embodiment of a growth matrix 3 suitable to support extra-particle mycelial growth, such as extra-particle aerial mycelial growth. The growth matrix 3 is shown as circles. In some embodiments, the growth matrix 3 can be contained within a tray 11 with a bottom and side walls as shown. The growth matrix 3 can include a growth medium and a fungus. For example, the growth matrix 3 can comprise growth media 2, substrate 1, and colonized (or pre-colonized) substrate 6 (which was inoculated), to support growth therefrom.

The growth matrix 3 can be exposed to environmental conditions imparted by environmental controls 15, contained either within the growth environment 16 or outside a growth environment 16 (the growth environment shown in dashed lines) contained within a growth environment system 12. The growth environment 16 may be a relatively small growth chamber, such as a benchtop bioreactor (BTBR) in which a relatively small amount of aerial mycelial material is grown, or a relatively large growth room, in which numerous large portions of aerial mycelial material is grown, such as in extended panels. One or more environmental conditions can be monitored within the growth environment via environmental condition monitors or sensors (aka environmental sensors) 19, which can either directly, or through a processor 17, communicate the need for changes in environmental conditions within the growth environment 16 in response to unsatisfactory condition changes, or timed condition changes to environmental controllers 15 (located either inside or outside the growth environment). For example, implementing condition change that affects the growth from the growth matrix for desirable end results. For example, a processor 17 and environmental condition controllers (aka controls) 15 can be provided with information from the environmental sensors 19, leading the environmental sensor 19 to implement environmental changes to control oxygen (O₂) content, carbon dioxide content (CO₂), other gaseous content, atmospheric pressure, temperature, humidity, misting rates, levels and times, misting liquid composition, misting direction, airflow direction and rate, light, and/or other environmental conditions to, from, and/or within the growth environment 16 (in order to establish base-line growth conditions). For example, such changes can be achieved through an interface 18, or a location within the growth environment 16. Environmental controls 15 may include HVAC controls/inputs, heating or cooling elements, misting apparatus, lighting apparatus, humidifying apparatus, nutritional addition apparatus, contaminant control apparatus, and such. Such changes in environmental conditions by the environmental controls 15 may include not only the periodic turning on or off of environmental systems once a desired condition is attained, but also the directing or redirecting of certain condition changes to certain locations within a growth environment, such as for example, the redirecting of mist nozzles or airflow in a growth environment to place aerial mist at concerning locations where it is deemed lacking or would be most useful for improved growth, based on the results of data (and/or comparisons with either system data for other locations in the growth environment, or previously referenced data for earlier growth runs, demonstrating normal trajectories for growth of aerial mycelium at that concerning location, and for that time period in the growth timeline). Other potential environmental condition controllers 15 may be used to introduce nutritional supplements or water into the system if deemed necessary or beneficial, such as to the growth matrix. The environmental condition sensors 19 may be traditional sensors such as thermostats, humidity detection apparatus, airflow velocity measurement devices, airflow directional measurement devices, gas detection devices, atmospheric pressure detection devices, light measurement devices, airborne liquid detection devices, contaminant detection devices, or nontraditional sensors such as infrared or heat imaging sensors.

In certain embodiments, the monitors and sensors may include image monitoring systems (e.g., optical, thermal, visual, infrared, near-infrared, acoustic, x-ray, chemical, and any other type of imaging), alternatively, visual monitoring devices, for example, image systems 20 (e.g., optical sensors or traditional cameras, including still image or video cameras). Image systems may include the following equipment: photodiodes and phototransistors, phototubes, photovoltaic cells, photonic sensors, fiber optic sensors, optical encoders, fluorescence sensors, reflective optical sensors, absorption spectroscopy sensors, interferometric sensors, lidar sensors, optical gas sensors, digital image sensors, mirrorless image sensors, digital single-lens reflex (DSLR) image sensors, action image sensors, compact image sensors, bridge image sensors, medium format image sensors, instant image sensors, 360-degree image sensors, web cameras, CCTV image sensors, thermal image sensors, trail image sensors, drone image sensors, film image sensors, smartphone image sensors, although any other suitable equipment may equally be applied. Such condition monitors 19 and image systems 20 may be singular in number or multiple, and may be placed at locations that are centralized for unobstructed monitoring such as in a central aisle, or throughout the growth environment 16 at locations such that essentially a monitoring array is present to communicate a representatively accurate sampling of conditions or growth levels throughout the entire growth environment from select locations. In certain embodiments, only image systems 20 may be present in single or multiple locations, rather than multiple types of environmental sensors within the growth environment 16, and changes in environmental conditions by environmental condition controllers 15 may be based solely on visual images (and comparisons with either other location images, or historical images for successful, similarly aged mycelial growth at that imaged location).

Such image systems 20 may be placed within the growth environment 16, or external to the growth environment 16, such as outside a window adjacent the growth environment 16, with ideally clear unobstructed view of the activities within the growth environment 16. The processor 17 may be singular or numerous as desired and may be connected to a network including multiple monitors, sensors (environmental condition sensors), image systems (optical sensors) 20, optional alert and reporting systems, and optional operational safety management systems. The environmental condition controllers 15 (and processors and human managers) may be further used, once base-line growth conditions are established/maintained for a desired period of time, to implement supplemental conditions (physical or mechanical manipulations, and/or chemical, biologic, or other substance additions) to the growth environment, apart from the base-line growth conditions. The supplemental conditions (physical action steps and substance additions) may be introduced for example, through various devices, apparatus, or channels 21, 22, 400.

In some embodiments, the growth matrix 3 is implemented without tray 11 (e.g., on another growth support structure, such as a planar support structure without side (or even bottom) walls, such as a mycological growth web, net, shelf, rack, or other supporting system, such as a bed. In some embodiments, localized misting apparatus 21 may be utilized to deliver alternative misting compositions including chemicals, biologics, and/or other additive substances. Delivery of moisture or other chemical, biologic and/or other substances/supplements may also be through drip or other irrigation means 22 to the upper surface, lower surface or internal portion(s) of the growth matrix.

FIG. 1B illustrates an embodiment of extra-particle aerial mycelial growth 8 from the growth matrix 3 of FIG. 1A. For example, the growth can occur when the growth matrix 3 from FIG. 1A is incubated or otherwise processed within a growth environment 16 under initially identified base-line growth conditions suitable for the growing fungal organism and the desired properties of the extra-particle aerial mycelium growth 8 in FIG. 1B. For example, in some embodiments, the environmental condition(s) within the growth environment can be controlled to induce substantially homogeneous extra-particle aerial mycelial growth from the growth matrix (which will be later separated from the substrate or matrix to produce aerial mycelium). Later introduced physical manipulations, as described below, may then be used as supplemental conditions to achieve an even higher level of homogeneity for the aerial mycelial growth outer surface (that surface facing the exposed atmosphere (such as the larger airmass) in the growth environment) than would otherwise be achievable with base-line growth conditions.

The extra-particle aerial mycelium growth can extend away from and outward from a surface of the growth matrix to form an aerial mycelium 7 as shown. Appropriate base-line growth conditions of the growth matrix 3 in FIG. 1A result in extra-particle aerial mycelium growth initiating across the exposed surface of the growth matrix. Next, extra-particle aerial mycelium growth continues to expand, forming a volume of extra-particle mycelial growth 8 as shown in FIG. 1B. The volume of growth can be generally contiguous, in that little or no gaps or holes in the extra-particle mycelial growth 8 occurs. The extra-particle aerial mycelium growth 8 can be grown to various heights. In some embodiments, the growth is about 3-4 inches high above the growth matrix 3. This can be achieved, for example, in up to about two weeks of growth, during which time the extra-particle aerial mycelium growth can become a mature mycelium. It will be understood that although the extra-particle aerial mycelium growth has some amount of irregularity to its upper surface topology as shown, the drawings are not to scale, and the top surface can be relatively flat when viewed from a certain distance. The extra-particle aerial mycelium growth 8 can be separated from the growth matrix at a position 9 (to form aerial mycelium), such that no growth matrix is transferred in the separation.

As noted, in some embodiments, the growth can be implemented on a mycological growth web, for example, without the tray 11 shown. The growth web can include the growth matrix and the extra-particle aerial mycelial growth (e.g., without a tray 11). The growth web can include any suitable support structure to support the growth matrix 3 and the extra-particle aerial mycelium growth 8, such as a net-like structure, or other perforated material. The web can be a standard size, such as a 63″W×38′L, 63″W×98′L or any of many other web configurations. Other sizes can be implemented, including lengths up to 90, 100 feet, or more. The net can comprise one or more layers of a perforated or nonperforated material, or combinations thereof, such as a plastic, nylon (e.g., nylon weave), or any other flexible, suitable material or multiple layers of material for growing extra-particle aerial mycelium growth 8 from a growth matrix 3. The web can extend in length from right to left in the orientation shown in FIG. 1B. As illustrated, the growth 8 demonstrates negative gravitropism, although positive gravitropism is also contemplated to be within the scope of the inventive methods and systems. Equipment modifications or additions 400 may be present in the growth environment to perform physical and mechanical manipulation/action steps to the growing aerial mycelium in order to trigger changes in growth levels, growth patterns, or growth trajectories, reduction or appearance in physical features, regrowth following damage (as described further below), and follow-up in situ physical manipulations and/or chemical, biologic, or other substance addition treatments in the growth environment.

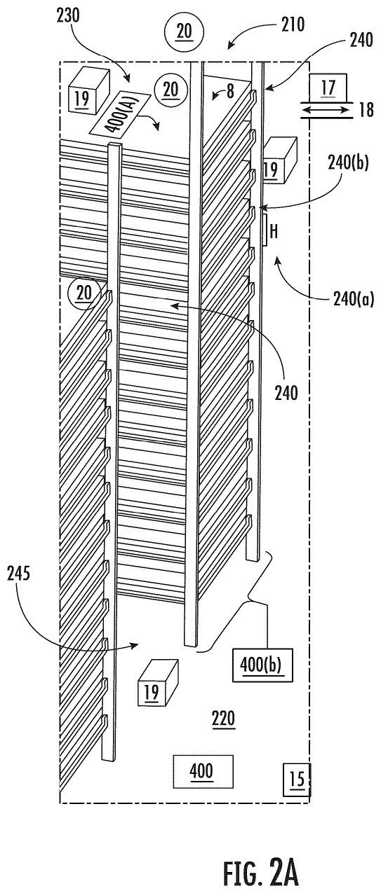

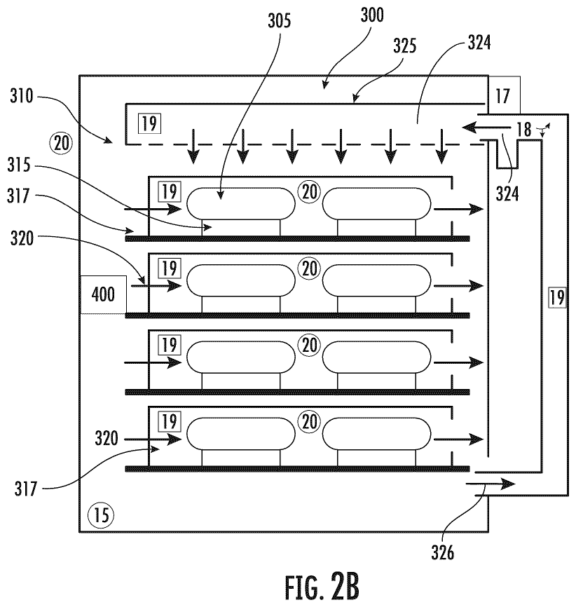

FIG. 2A illustrates an embodiment of an isometric view of an aerial mycelium growth environment system 210 for cultivating aerial mycelia within a growth environment 220, such as in a larger growth room or chamber. It should be understood that each of the methods and systems described herein can be applied to growth environments of a significant size. The growth environment 220 can be configured to grow aerial mycelium from a growth matrix positioned therewithin. For example, system 210 and growth environment 220 contained therein can be implemented to facilitate the aerial mycelial growth described above with reference to FIGS. 1A-1B and can include similar components and functionality.

The growth environment 220 can include one or more shelves 240 (e.g., vertically configured shelves), on which a growth matrix can be positioned, and from which extra-particle mycelial growth can extend. The growth matrix (or alternatively, inoculated substrate) can be positioned directly on a shelf, or with an intervening growth support structure, such as a growth web, net, or bed. One or more racks (or shelving units) 230, such as the two racks shown separated by an aisle 245, can include a plurality of the shelves 240 (e.g., stacked vertically), positioned within the growth environment 220. The environment can include various numbers of racks, which can include various numbers of shelves 240, and can be of various dimensions. For example, two or more racks 230 can form two sets of shelves 240, wherein each shelf in each set can be positioned at approximately the same height as a corresponding shelf in the other set of shelves. A shelf can be sized and configured to support a web, net, bed or other growth support structure, such as those described above with reference to FIGS. 1A and 1B, or even other growth support structures. In some embodiments, the racks include 12 shelves and are 12-16 feet high. The shelves 240 can move vertically to compensate for spacing shelves 240 at different heights. The shelves 240 can be spaced apart from each other (e.g., vertically) a spacing height H that is sufficient to allow for the height of the desired extra-particle aerial mycelial growth combined with the height of the underlying growth matrix, sufficient clearance for airflow, mist flow, processing, and handling. It will be understood that air, air content and airflow as used herein can refer to various compositions of various gases, and flow of those gases suitable for the products and methods described herein and should not otherwise be limited to a particular composition or ratio of gases. Additionally, some embodiments herein that are described as being implemented with respect to “water,” such as in connection with mist, may be similarly implemented with other liquids.

The spacing height H is defined as the distance between the same two corresponding points on two adjacent shelves 240. For example, the spacing height can be defined as the distance from the top surface of a first lower shelf 240(a) to the corresponding top surface of an adjacent upper shelf 240(b). In some embodiments, the spacing height can be in a range between about 200 mm to about 530 mm, or between about 225 mm to about 490 mm, or between about 250 mm to about 450 mm. In some embodiments, the spacing height can be less than 530 mm, less than 490 mm, or less than 450 mm. In some embodiments, the spacing height can be about 350 mm. These spacing heights can be advantageous because the nature of the aerial mycelial growth herein requires less spacing, and thus can allow for an increased number of shelves and higher output than conventional mushroom cultivation. Such a growth environment 220 can include one or more environmental condition controls 15, one or more environmental condition sensors 19, and one or more image systems 20 (optical sensors such as image sensors), each designed to specifically encompass/monitor either a desired portion of the growth environment or the entire growth environment 220. Such sensors, such as the image systems 20 may be positioned within the growth environment 220 or outside such environment, such as adjacent a glass window in a wall defining the growth environment. Shown in abstract forms, one or more various equipment modifications or additions 400 may be made within the growth environment 220, to provide or impart physical manipulation or physical action steps to one or more portions 400(a) of individual substrates, growth matrices, or growing aerial mycelium (such as the one shelf portion shown on the uppermost shelf of 240), or alternatively to portions of the growth environment 400(b) as a whole, such as one of the shelf units 240 in a growth environment. For instance, such equipment modifications or additions (including for example, individual rack hardware apparatus), may be positioned to traverse up and down individual shelves either horizontally or vertically, in order to impart physical or mechanical manipulation/physical action upon portions or the entirety of one or more beds or trays contained on shelves, or select locations of beds, or even to select portions of entire growth chambers making up a growth environment. Such hardware may include trolley-like systems, moveable rolling systems mated to shelf hardware, or drone applicators, each of which are capable of applying select physical manipulation actions or mechanical manipulation to one or more portions of materials or locations within growth environments. Hardware for introducing chemical, biologic, or other substance addition to impart desired supplemental growth conditions, may also be temporarily or permanently affixed to shelves or other locations within a growth environment, or applied via non-affixed and portable devices such as drones, and subsequently activated as desired to alter the growth trajectory (such as to alter or enhance particular growth runs) of one or more portions of either individual beds, trays, or webs of inoculated substrates, growth matrices or growing aerial mycelium, or alternatively one or more portions of the growth environment itself (such as whole shelving units, portions thereof, or portions of the larger atmospheric void space within the growth environment). Such chemical, biologic, or other substance additions may be introduced also via base-line growth condition equipment, such as for example, HVAC, or misting apparatus as previously noted.

FIG. 2B illustrates an embodiment of a system 300 for creating a growth environment 310 for growing aerial mycelium 305, such as on a commercial scale. The example of a system 300 for creating a growth environment 310 for growing aerial mycelium 305 on a commercial scale, shows a schematic view of a growth environment or growth chamber 310 having a series of trays 315, or beds on racks on shelves 317 contained in the controlled environment in which airflow 320 is directed horizontally across each of the trays, containers, or beds after entering the chamber through a manifold 325. In the illustrated schematic, airflow enters the manifold at an entrance 324, travels through the manifold and is directed to one side of shelves 317 where it is allowed to enter discrete spaces 320 and pass horizontally over the surfaces of inoculated substrate, growth matrix and/or aerial mycelium growing therefrom 305. The direction of airflow is illustrated throughout the schematic by the directional arrows. It may exit at 326 and be reintroduced and redirected to target locations across the growing aerial mycelium and/or substrate or matrix, as previously described.

Environmental condition monitors/sensors 19 are placed about the growth environment 310. As with prior system embodiments, a processor 17 is present to communicate with one or more optional environmental condition monitors 19, one or more environmental condition controllers 15, and one or more image systems 20 to control conditions within the growth chamber 310 in response to observations from said monitors, 19, 20. The numbers and placement of the monitors may be few or numerous, based on the homogeneity of conditions throughout the growth chamber, and/or the capabilities of the chosen sensors/monitors. In one embodiment, the range of monitors include both image systems 20 to periodically monitor the actual growth (such as for example current growth condition and/or calculated growth rate based on a series of compared images and algorithmic calculations) of aerial mycelium off of the substrate or growth matrix), and environmental condition monitors 19, with both types of monitors communicating with a processor or a network of processors as may be desirable (which are either centralized or dispersed and with either centralized or dispersed operational models associated therewith).

The various sensors in the growth environment 310 communicate/transmit data/images to the one or more processor(s) which may run one or more associated evaluative models (based on previously loaded data management systems). Based on the one or more various models, adjustments may be made as needed to the environmental conditions via the environmental condition controller(s) 15 in the growth environment 310 in order to achieve a targeted aerial mycelial growth level and type, for the time period desired, or alternatively, to make up for immature growth in particular locations within the growth environment 310, or still alternatively, to identify and properly address permanently deviant, temporarily irregular, or unexpected aerial mycelium morphology which may be present in the growth environment 310. As seen in FIG. 2A, equipment or apparatus modifications 400 may be introduced to the growth environment shown in FIG. 2B to allow for physical or mechanical manipulation or physical action (introduction of supplemental growth conditions) upon select portions of materials (such as contained on individual trays or beds), shelving units, or environmental void space contained within a growth environment. Similarly, chemical, biologic, and/or other substance additions as supplemental growth conditions may be introduced through previously described apparatus/technology.

Aerial mycelium growth system architecture including equipment and apparatus designed to physically and mechanically manipulate or impart physical action to one or more portions of substrate, growth matrices, growing aerial mycelium, may be organized according to one embodiment of the invention. The system architecture may include a network, in communication with one or more optical sensors, one or more optional environmental sensors, controller elements of an environmental control system, optional alert and reporting systems, to communicate select alerts to human managers of the system and log status of conditions generating alerts, optional operational safety management systems to effectuate steps to address safety issues that may occur within the system architecture, record entries of specific details and prepare and transmit related communications. Environmental control systems can be configured for implementing action steps, including physical manipulation action steps and substance addition (to impart supplemental environmental growth conditions) to alter base-line environmental growth conditions relating to for example, HVAC (airflow, airflow velocity, airflow rate, airflow direction, and airflow heating and/or cooling), gas content (such as by off-gassing if necessary undesirable gases), mist deposition (such as to initiate airborne liquid mist, regulate such mist levels, mist composition and/or direction, discontinue such mist, redirect such mist, either in periods by timing, or as needed, to provide for example, increased moisture or nutrient levels), light levels, relative humidity, substrate composition (such as moisture and nutritional levels) and such. For example, one action step can include one or more compositions of mist from one or more mist sources (e.g., various mist-containing, nutrient formulations available to one or multiple locations within a growth environment). In some embodiments, the action steps to modify one or more base-line environmental growth conditions can be implemented by at least one of a temperature control device, a mist control device, a relative humidity control device, an atmospheric pressure control device, and an atmospheric gas control device, an airflow control device, or any other device that relates to HVAC, gas content, mist deposition (including to initiate airborne liquid mist, to regulate mist levels, composition and/or direction, to discontinue mist, to redirect mist), light levels, relative humidity, substrate composition or any other environmental condition. For example, a mist control device can be configured to control misting levels, misting liquid composition, misting direction, or any other misting action. A mist control device can be configured to selectively introduce chemical, biologic or other substances where and as desired, in order to produce enhanced attributes or targeted attributes to one or more growing aerial mycelium panels within a growth environment or portions thereof (which enhanced or targeted attributes differ from those of neighboring growing aerial mycelium panels in the growth environment, as a result of those neighboring panels only being exposed to base-line growth conditions).

The various monitors, sensors, or devices that may be positioned in and around the growth environment may include for example, one or more optical sensors (such as RGB image sensors, photographic, or videographic sensors), and environmental sensors (such as temperature sensors, humidity sensors, infrared image sensors, thermal image sensors, air monitors, mist visibility monitors (including monitoring mist liquid compositions and/or direction), light monitors, gas monitors etc.) A set of sensors may be distributed throughout the growth environment to sense data at various locations throughout the growth environment in an array or lattice configuration, such as one or more for each shelf or rack or bed, or centrally located sensors, strategically placed in locations that are representative of conditions throughout the whole growth environment. The optional operational safety management system may maintain the general operational safety program for the growth environment. In some embodiments, the operational safety management system keeps records related to operational safety for the growth environment. The optional alert and reporting system monitors, logs, and reports the operations of the system elements, the one or more types of sensors, and the software running in the system (such as in the processor(s) and network).

As noted with respect to United States Patent No. 11,277,979 to Greetham et al., International PCT Patent Application No. WO2019/099474A1 to Kaplan-Bie et al., PCT Patent Application No. WO2023172696 to Snyder et al., PCT Patent Application No. WO2022235688 to Winiski et al., and PCT Patent Application No. WO2022235694 to Carlton et al., certain base-line growth conditions have been described in the cited references for generating aerial mycelial growth. So as to implement supplemental growth conditions (aka supplemental environmental growth conditions), additional systems or apparatus may be provided in and about growth environments to either introduce new chemicals, biologics, other additive substances or materials to inoculated substrates, growth matrices or growing aerial mycelium materials. Such supplemental growth conditions may be applied to discrete portions of substrates, growth matrices, or growing aerial mycelium materials, such as discrete portions of individual trays or beds, or portions of materials contained in growth chambers, or alternatively across entire chambers, to enhance or alter growth of materials across an entire chamber. Similarly, discrete hardware, such as trolley or drone devices may be implemented in accordance with the invention, to perform physical manipulation or action steps upon select portions of substrates, growth matrices, or growing aerial mycelium material, while optionally leaving neighboring substrates, growth matrices, or growing aerial mycelium material untouched (and therefore only exposed to base-line growth conditions).

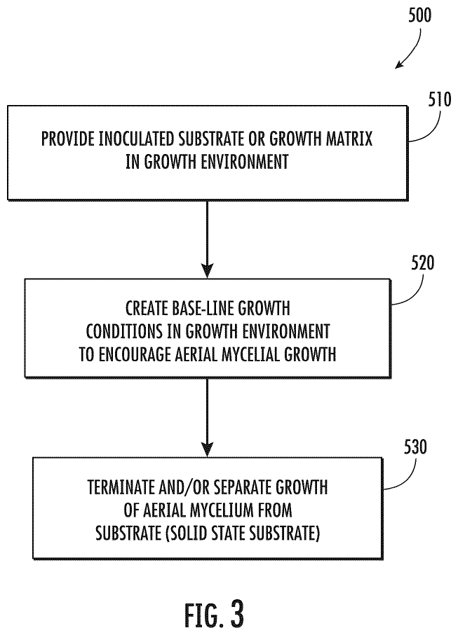

In order to illustrate various methods and processes for growing aerial mycelium in accordance with the invention, various flow diagrams are provided in FIGS. 3-6 to demonstrate a series of supplemental process steps (supplemental conditions) that may be practiced to alter or enhance the growth trajectory of aerial mycelium grown through solid state fermentation systems, when compared to aerial mycelium grown under base-line growth conditions. So as to provide a basis for comparison, an initial method for growing aerial mycelium in accordance with only base-line growth conditions 500 is shown through a series of process steps in FIG. 3. As seen in the method/process 500 of FIG. 3, a fungal inoculated substrate (or growth matrix) is provided initially in a growth environment 510. Following the loading of the growth environment with the selected fungal inoculum and growth substrate (or growth matrix), a series of base-line conditions is established in the growth environment 520 to actively encourage the occurrence of aerial mycelial growth. As has been described in the cited references for instance, such conditions include desirable levels of humidity, mist, gaseous environment, airflow, temperature, light, and available nutrition. In such a base-line condition environment, aerial growth occurs and then is eventually or optionally terminated (that is, rendered metabolically inactive and/or the aerial mycelium is separated from the substrate).

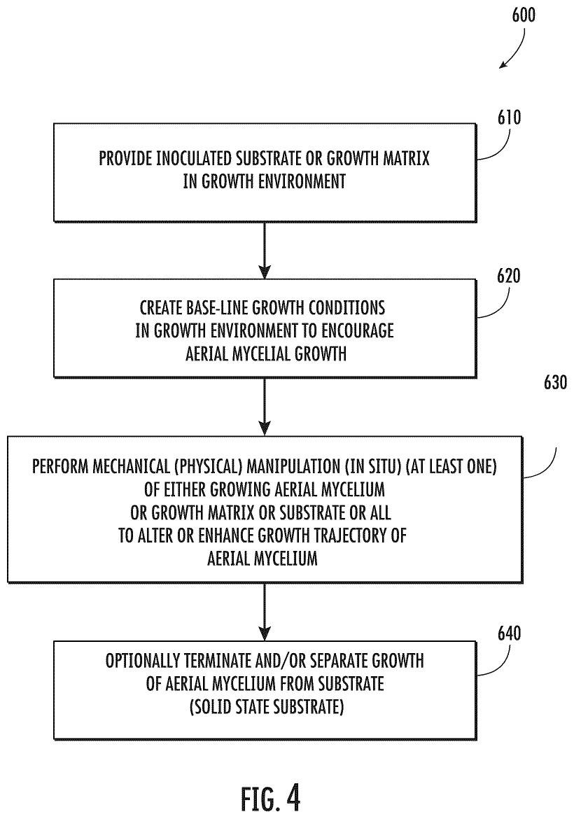

So as to change the growing or grown aerial mycelium material from that which may be grown under only base-line conditions, one or more additional process steps may be implemented to act on either the inoculated substrate, growth matrix, or the growing mycelium material (or a combination thereof), to either change the final resulting grown aerial mycelial material (so as to enhance one or more material properties of the grown aerial mycelial material or to impart one or more new properties or attributes to the grown aerial mycelial material), or to expedite the growth of grown aerial mycelial material (or a combination of one or more of these purposes). The additional process steps may be implemented in a variety of numbers, orders, and/or combinations (with or without temporal periods between them), but for the purposes of simplicity, they are illustrated in FIGS. 4-6 in specific process/method sequences. Such specific sequences and numbers of process steps should not be interpreted as being limited to those shown, unless otherwise stated. It should also be recognized that at a given time, both a mechanical action and substance addition (that is one or more of, or either a chemical, biologic, or other additive) may be implemented in the aerial mycelium growth environment. Alternatively, such conditions may be implemented separately, and spaced apart by a temporal delay.

As shown in FIG. 4, a method of growing aerial mycelium using a solid-state fermentation process is represented by the flow diagram of 600 with a series of process steps shown. The method includes a first process step of providing inoculated substrate (or a growth matrix) in a growth environment 610. As described in various cited references herein, the inoculated substrate is a solid-state substrate and the environment can be an environment suited to achieve the growth of aerial mycelium, such as for example, the controlled setting of a bench top bioreactor, a growth room, or chamber. Once securely housed within the growth environment, the inoculated substrate is exposed in a second process step, to a base-line set of growth conditions designed to encourage aerial mycelium growth 620 from the inoculated substrate or growth matrix in the growth environment. At a time following the creation of base-line growth conditions within the growth environment, one or more supplemental conditions, in the form of a physical or mechanical manipulation process step(s) is performed in situ within the growth environment to either the inoculated substrate, growth matrix, or the growing aerial mycelium 630 or a combination, which mechanical manipulation step(s) either enhances or alters the growth trajectory of the aerial mycelium, such that the thereafter grown aerial mycelium either demonstrates enhanced properties (or attributes) or different properties from those which would have been demonstrated experiencing purely base-line growth conditions, or alternatively, experiences more rapid growth as a result of such manipulation(s). Examples of such physical manipulations include but are not limited to, physical contacting steps of the growing aerial mycelium with an apparatus designed to directly or indirectly physically impact, move, or redistribute the aerial mycelium material, such as a roller or other compression tool, a blade, and a fluid-directing device (such as an air-directing fan). Examples of other physical manipulation devices may include environmental stimulation devices that alter the trajectory of growth of the aerial mycelium, such as an electric field or sound wave introduction in all or a portion of the growth environment. Additional physical manipulations may include actions which temporarily bruise or damage the growing aerial mycelium (actually extra-particle aerial mycelium), or which help or encourage layering of such material (such as in an action of folding it). Such mechanical or physical manipulations may be done multiple times, sequentially, or at discrete separate temporal periods, and may include different types of manipulations. Following the one or more physical manipulations, the aerial mycelium growth is optionally terminated or separated from the substrate 640 in an additional process step.

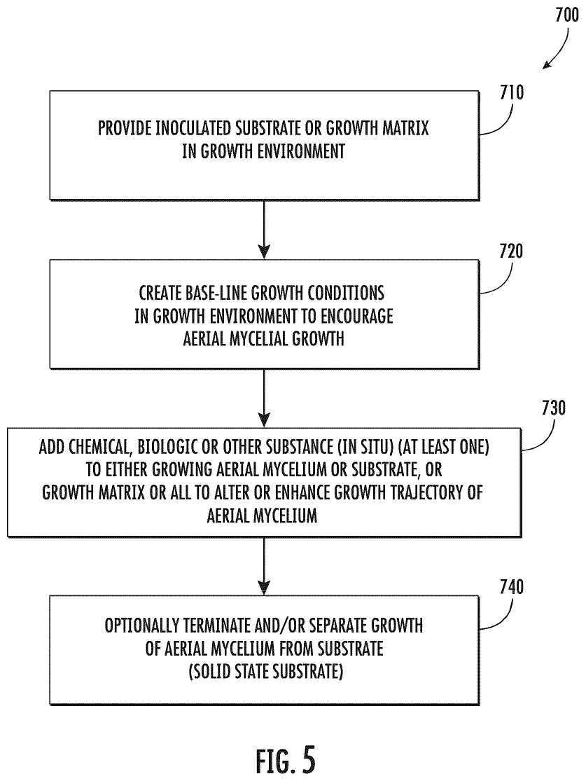

In an alternative method as shown in FIG. 5, a process for growing aerial mycelium includes the addition of either a chemical, biologic (al), or other additive substance to either the inoculated substrate (alternatively, a growth matrix) or the growing aerial mycelium, in order to alter or enhance the growth trajectory of aerial mycelium, beyond that which would have been achieved using just the base-line growth conditions 700. The process includes the step of first providing inoculated substrate (or alternatively a growth matrix) in a growth environment 710. Following the provision of inoculated substrate (which is solid-state substrate), base-line growth conditions are created or established in the growth environment to encourage aerial mycelial growth 720. Upon the establishment of base-line growth conditions, a chemical, biologic(al), or other substance (or multiple chemicals, biologics, or substances) is/are added in situ to either the growing aerial mycelium or substrate (or both), to alter or enhance the growth trajectory of the aerial mycelium 730. Such addition(s) may be done multiple times, sequentially, or at discrete separate temporal periods. Different substance(s) may be applied, or multiple applications of the same substance(s) may be applied. Upon the growth of such enhanced or altered aerial mycelium, the growth may be optionally terminated and/or separated from the inoculated substrate. The sequence of addition of the chemicals or other substances may vary by the aerial mycelium product desired or the attributes desired.

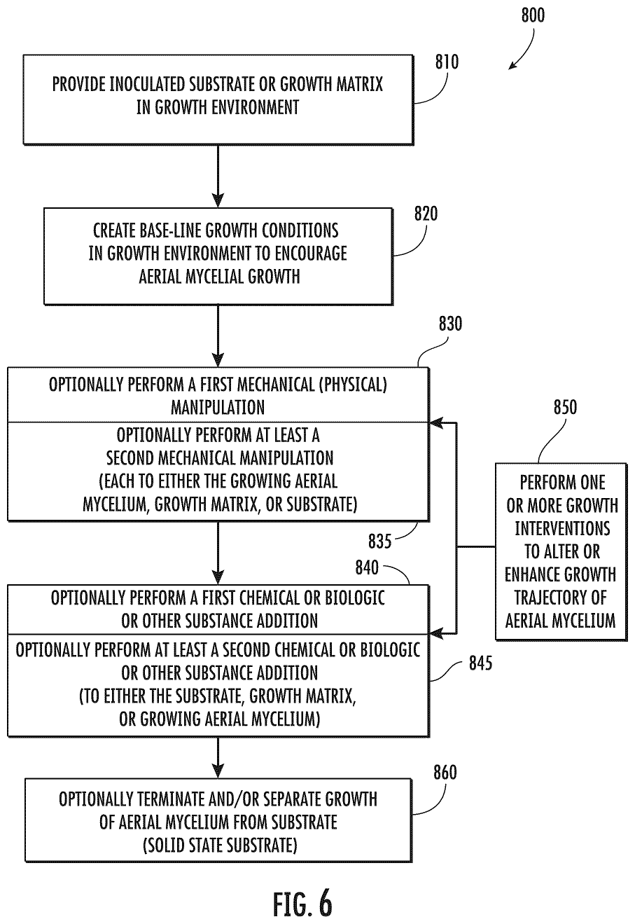

In yet another embodiment of a process in accordance with the invention, and as seen in FIG. 6, multiple optional mechanical (or physical) manipulations and/or optional chemical/biologic/substance additions may be implemented to the growing aerial mycelium and/or inoculated substrate (or alternatively growth matrix if included). As seen in FIG. 6, a process 800 is illustrated in which both optional physical or mechanical manipulations and optional chemical/biologic/substance additions are contemplated. For instance, the process may include one of each a mechanical and chemical addition, or two or more of each mechanical and chemical additions. Actions may be concurrently applied or spaced apart in time.

As illustrated, in a first process step 810, an inoculated substrate or growth matrix is provided in a growth environment. Following the provision of the inoculated substrate or growth matrix in the growth environment, a baseline set of growth conditions is established in the growth environment to encourage aerial mycelial growth 820 (and in particular extra-particle aerial mycelium growth since the substrate is solid-state). Following the establishment of the base-line conditions, optional physical or mechanical manipulation steps (one or multiple) are performed upon either the inoculated substrate, growth matrix, or growing aerial mycelium 830, 835. Either following the optional mechanical manipulation steps, or preceding the optional mechanical manipulation steps, or concurrently with the optional mechanical manipulation steps, one or more optional chemical, biologic, or other substance addition steps are also conducted to either the inoculated substrate, growth matrix, or growing aerial mycelium (or both) 840, 845. Upon completion of one or more optional physical or mechanical manipulation steps or optional chemical/biologic/substance addition steps, the growth of the aerial mycelium may be optionally terminated (that is rendered metabolically inactive) and/or separated from the substrate 860 in a process step. It should be recognized that for this method, while the sequence is shown as first including mechanical manipulations followed by chemical/biologic/other substance additions, the method is not meant to be limited in such fashion. The supplemental conditions may be implemented in a variety of sequences and timing periods. They may be implemented at the same time, or at different times, and they each may be repeated as desired.

So as to further elucidate various physical manipulations/action steps and chemical/biologic/substance additions that are contemplated in accordance with the invention, the following examples of supplemental growth conditions are now provided.

### EXAMPLES OF SUPPLEMENTAL GROWTH CONDITIONS IN ADDITION TO BASE-LINE GROWTH CONDITIONS

### Example of Chemical/Biologic/Other Substance Addition-Supplemental Growth Conditions

### Example 1

In a first example, substances are applied either to an inoculated substrate, growth matrix, or growing aerial mycelium (or portions thereof), through either an in situ drip system 22 or mist application system 21, such that the addition is either ingested or absorbed into growing aerial mycelial tissue (and incorporated therein) or coated upon growing aerial mycelial tissue (or substrate or matrix) in layers. Such additions may include for example: (1) amino acids, amino acid derivatives (Creatine, etc.), (2) water soluble vitamins (B1, B2, B3, B5, B6, B7, B9, B12), (3) guanosine and inosine monophosphates, (4) thiamine, and (5) salts, such as for instance calcium chloride and sodium hydroxide.

### Example 2

In a second example utilizing the technique of in situ mist deposition, applied mist includes a mild acid, such as acetic or citric acid, which can be applied throughout or at an interval of the aerial mycelium growth run. Such a mild acid can solubilize glucans in the extracellular matrix of the growing aerial mycelium (which in one embodiment, may occur towards the end of the designed growth run).

### Example 3

In a further example of addition of a chemical substance to the growing aerial mycelium material in situ, application of pigments and/or liquid nutrition may be implemented either directly to the growing mycelium material surface or through the inoculated substrate such as through 22, 400, to be introduced to the growing fungal organism. Such can be implemented either with or without compression on the growing aerial mycelium. If in conjunction with compression, pigments or liquid nutrition may be applied to a roller prior to compression, then allowing for compression and deposition of the liquid media. Or alternatively, a pressure roller may be perforated and liquid media may be extruded through the roller surface to print concurrently with compression. Still further, the roller may include small-or large-gauge needle projections to inject substances (liquid, gas, emulsion, or suspended solids) into the aerial mycelial tissue while rolling. An array of injection needles may also be applied by a flat plate as an alternative to a roller. Alternatively, chemical growth inhibitors and/or promoters may be applied either to the growing aerial mycelium material, inoculated substrate, or growth matrix. Such may be applied as noted through either drip irrigation or mist applications for example, either continuously or through temporal variations. In still a further example, such chemical applications may be designed to encourage additional branching of fibers. Such may include cross linking agents or employ pH modifiers, and/or calcium.

### EXAMPLES OF IN SITU INTERVENTION IN THE FORM OF PHYSICAL OR MECHANICAL MANIPULATION/PHYSICAL ACTION STEPS AS SUPPLEMENTAL GROWTH CONDITIONS

### Example 4

In a further example, a periodic compression is applied to the growing aerial mycelium material at either regular or dynamic intervals in situ upon the achievement of desired observed growing aerial mycelial thresholds. The compression may be applied via physical means, electromagnetic attractive/repulsive forces, air velocity, plates, or rolling techniques.

### Example 5

In example 5, the growing aerial mycelium material may be exposed to tangential application of force (such as for example dragging of a contacting surface or applied air velocity) to orient fibers along one or more of its dimensions or to effectively create an abrasion upon the tissue. The tangential application of force may be applied periodically or continuously for a period of time, such as by the dragging of a wet thread or string across its surface. Alternatively, the material may be impacted by air weaving in which periodic bursts of air or other gas or fluid, forces fiber reorientation, overlapping, or weaving. Still further, such fibers may be exposed to needle punching.

### Example 6

Microperforation of the growing material in a patterned or random manner can also allow for air perfusion from an aerated bed by introducing gas under the substrate and allowing it to rise through the growing material via the microperforated channels. The movement of gas could cause the channels to expand by force alone, if the gas is inert, or induce some metabolic or otherwise structural changes in the aerial mycelium if the gas is reactive or metabolically significant, such as oxygen or carbon dioxide. The gas could be humidified or controlled in temperature for desired changes.

### Example 7

In a further example, the growing aerial mycelium material may utilize additional materials in the in situ growth process, such as multi-ply or particulate tissue material for regrowth or adhesion as a composite material itself. In yet another embodiment of this utilization of additional materials example, such composite applications may utilize additional growth substrate including powdered nutrients, substrate, or non-viable mycelium material. As still a further alternative of utilizing additional materials in the in situ growth process, additional viable aerial mycelium materials may be incorporated into the growth process, such as aerial mycelium from remote growth environments and aerial mycelium resulting from the same growth operation (such as from an earlier aerial mycelium growth run). As a further example of a utilization of viable aerial mycelium material in a growth process, such material (for example, partially obtained from prior or concurrent growth runs) may be rolled, cut, and then stacked upon other growing aerial mycelium sheets or panels, with orientations of the rolled fibers of each layer alternating in different directions, such as for example at 45 degrees, 60 degrees, 90 degrees, etc., in order to produce a final grown aerial mycelium material with desirable attributes.

### Example 8

In still a further example, the physical manipulation or physical action steps may consist of directed “damage” to one or more portions of the growing aerial mycelial material, or periodic tensioning on the growing aerial mycelial growing tissue, in order to induce ligative/anastomosis (as a muscle-like bioreactor). Alternatively, damage by rupturing or slicing/scoring the surface of the material would impact the growing surface by both introducing openings and additional surface area for growth or subsurface introduction of substances. Slicing or scoring of the material also may be used to normalize the density of the material by disrupting bulbs or dense regions of the growing aerial mycelium.

### Example 9

In yet a further example, a roller with strings attached to it, and spinning above the surface of the growing aerial mycelium may be used to drag individual tufts of fungal hyphae of the growing aerial mycelium down into a relatively more horizontal orientation. The hyphae may stick to the string via surface tension and be left agglomerated in large clumps by a surface tension effect. Alternatively, the roller may instead be a disk-like configuration, which may move across the surface, producing an entanglement effect, and generating a horizontal non-woven motif of agglomerated structures, aside from what fibers may be grown natively. In still a further alternative of a roller compression embodiment, a compression action combined with pattern embossing functionality, such as via a roller positioned over the in situ growth bed (or a platen-like press positioned over the bed) may be utilized, in which either the roller or platen functions as a die to impart a defined pattern in addition to partial or overall compression. Such configuration is available for example, from Ivan Leathercraft under the Speedy Embossing Machine.

### Example 10

In yet a further alternative embodiment of mechanical manipulation techniques (involving growth-responsive physical intervention according to any of the above exemplified methods), such physical-action responsive growth would be observed by sensing devices (camera, range finder, etc.) and the physical intervention is operated at defined growth run intervals during observation. For example, roller compressing is applied in one example, at every 2 mm of growth progression or in a defined sequence (for instance, (Step 1) 2 mm growth followed by (Step 2) rolling, followed by (Step 3) 6 mm growth, followed by (Step 4) rolling etc.).

Alternatively, the material may be periodically compressed or rolled in a growth bed during growth, to redirect actively extending hyphae so they entangle and then continue to grow. A shim may be positioned above the substrate and alongside the tray/bed edge holding the inoculated substrate.

An adjustable roller can, in still a further example embodiment, periodically knock down surface growth a certain percentage of its overall growth height, such as for example 10% of its overall height, daily or at some other desired temporal period. Still further, the roller may introduce any advantageous texture or geometry in doing so. Still further, such a roller may roll in such a fashion to promote or restrict edge growth or dimple the surface to enhance gas exchange within the growing aerial mycelium material.

### Example 11

In still a further example, mechanical manipulation may be used to simulate the actions of (and lead to the resulting material responsive stress of) insects foraging in natural mycelium (which naturally differs from aerial mycelium of the present invention) to stimulate sub-apical branching and anastomosis. In still another insect-related example embodiment, the inoculated substrate may be inoculated with fungus beetles. In still a further alternative embodiment, insect-like micro or nano-robots may be used upon the growing aerial mycelium material to simulate insect movements upon the developing aerial mycelium surface, in order to simulate physical interactions with insects foraging on mycelium in nature, recognizing the above distinctions (and the resulting damage and regrowth caused thereby).

### Example 12

In yet another example, a vibrating or oscillating needle punch (such as a bed of needles) is punched into the growing aerial mycelium material, followed by vibration or oscillation of the needles to cause tearing of hyphae, and to eventually stimulate a damage response of the growing aerial mycelium, leading to sub-apical branching and anastomosis as a strategy for creating redundant translocation pathways. Still alternatively, a needle punch may be applied to growing aerial mycelium material and left in place for enough time to allow for further aerial mycelium development (in contact with or punched into the mycelium bed), then removed after regrowth to cause a damage response.

### Example 13

In still a further example, a non-nutritive sheet is placed over an inoculated substrate bed and incorporated into growing aerial mycelium during growth. Periodically, tension may be placed on the sheet to deform, stretch, or damage the developing aerial mycelium to induce a damage response. Alternatively, the sheet may be electrically or thermally conductive, allowing for periodic electrification or heating of the developing aerial mycelium.

### Example 14

In still another alternative embodiment example, distributed metal or other solid, non-bioreactive material pieces (e.g., ball bearings) can interact with the surface of the growing aerial mycelium bed (such as by being rolled across) to create material damage for eventual re-growth, or may be manipulated by electromagnetic fields to move and create damage, or be allowed to embed within tissue for damage, and in either event, eventually collected and re-deployed for later use. The mode of applying compression could be uniform or highly variable in its spatial distribution. As an example of uniform force, a solid or perforated metal plate or mesh could be applied to the growing surface and compressed against by electromagnetic repulsion from an above source, then removed by electromagnetic attraction to the source. Metal marbles or ball bearings could also be deployed across the growing mycelium surface and manipulated along the plane through electromagnetism. The rolling action could apply one type of constant force due to gravity as well as additional compressive force at a constant rate or at specific points across the material by inducing an electromagnetic field. Collection of the metal objects could then be accomplished by electromagnetism as described above.

### Example 15

In still a further alternative embodiment example, sound, in place of, or in combination with airflow may be used to disrupt or modify aerial mycelial growth in situ in the growth environment. In this embodiment, sound waves are directed at the growing aerial mycelium tissue. The sound waves may be of an intensity such that they influence the growth of the fibers either by influencing the growth at a cellular level, or by actively manipulating entire hyphae or hyphae strands, by causing them to vibrate back and forth, for example, by displacing themselves at least about a 0.01 hyphal cell wall thickness, alternatively at least about a 0.1 hyphal diameter, alternatively between about 0.01 hyphal cell wall thickness to about 0.1 hyphal diameter, from their resting point. Such displacement may lead to a more intentional growth orientation.

In an alternative embodiment using sound, a speaker may be positioned above the aerial mycelium fibers, such as a distance of about 5-7 inches, or alternatively about 6 inches above them. Tones are then played on the speakers to produce a standing wave in the aerial mycelium fibers, such that they vibrate back and forth in space, creating a standing wave similar to the frequency selected on the speaker. This vibrating pattern is then reflected in the growth of the aerial mycelium tissue as each new level of hyphae form. In yet still a further alternative, frequencies are played as they would be played to a flat surface creating standing waves or patterns in sand. In the case of mycelium, each standing wave becomes a grown layer of mycelium, with the next layer reflecting the same wave pattern or a different wave pattern. In this fashion, extremely fine patterns of aerial mycelium can be formed in situ.

In this same manner, sound waves can be used to deposit or manipulate small particles or micro-beads placed on the growing aerial mycelium, either to deposit them in locations or distributions as desired, or once they are deposited, to trigger some other action of the particles or micro-beads.

In yet another embodiment, the sound waves are not employed to create standing waves, but instead create planned or random “walks” or movement of the growing aerial mycelium fibers, “vibrating” them around each-other to increase the probability of interference and fusion of aerial mycelium fibers.

In additional embodiments employing sound waves, speakers are placed above the inoculated substrate. In others, a single subwoofer is placed in the growth environment (such as a growth room or chamber). Still in further embodiments, a mechanical vibrator, such as a cement vibrator, is placed on a rack, shelf, or bed holding an inoculated substrate and growing aerial mycelium, in order to transfer vibrations to the inoculated substrate and/or growing aerial mycelium itself.

In most of the above-described cases, frequencies between 0.1 Hz and 100,000 kHz may be used. These can be receptive pattern, pattern sweeps, “funky” beats, and or patterned sequences that are reflected in the growth patterns of the aerial mycelium.

### Example 16

In yet still another example, re-growth of multiple discrete aerial mycelium layers into a laminate composite structure may be achieved in a single growth environment. Aerial mycelium material may be produced on multiple adjacent beds, cut from their respective inoculated substrates, then laminated together into an assemblage (where the bottom most layer may remain attached to its inoculated substrate to provide a continued nutrition and water source). Further incubation may follow, continuing to allow for fusion of the still growing aerial mycelium layers (assuming the same genetic individual self-self recognition will allow for fusion via anastomosis as well as general hyphal entanglement). Physical intervention per above, may be applied to the individual layers prior to assembly or after assembly of multiple layers, after which re-growth is then allowed to occur in the growth environment.

### Example 17

In yet still another example, a deconstructed ‘mesh’ (i.e., monofilaments as found in fishing line) can be extended across the top layer/upper surface of growing aerial mycelium material (perpendicular to an edge of a growth bed for example), and then manipulated after overgrowth of the mesh has occurred (such manipulation including such action steps as vibration, translocating at angles, etc.) and subsequently retracted, alternatively, raised in the growing direction, and repeated. If each mesh filament is spaced, for instance, 6 inches apart across and along the entire bed length, alternating physical manipulations could produce a regular repeating pattern in the z-direction of the growing aerial mycelium material. Depending on the diameter of the mesh filaments employed, a larger void space could be created with inflatable or stent-like designs. The void space created could also create physical/morphological differences at the tissue interface in the interstitial space.

### Example 18

In still a further alternative example, sensors may be incorporated directly into any of the aforementioned physical manipulation (and/or substance addition) tools, each offering the opportunity for real-time sensing and information capture, beyond imaging. These data collected at the point(s) of intervention would inform future manipulation and/or addition throughout the growth cycle as a feedback loop.

### Example 19

In another example, the use of drones may assist in imparting/conducting any of these physical interventions. Alternatively, drones may assist with air movement/airflow intervention, either to direct airflow or provide for pressure/air impacts. Alternatively, a fleet of small drones may be deployed in a pre-programmed manner to land, aerate, and take-off from one or multiple locations in a single bed or entire growth environment (chamber or room) offering more flexibility and potentially more opportunities for efficiency than physically connected tools for manipulation. Alternatively, these drones can be modified with attachments to needle punch, create depressions for water pooling, etc. Additionally, cargo or payloads carried with these drones can also be deposited on growing aerial mycelium surfaces in situ. In still a further alternative, claws or other moveable drone extensions either directly on the drone body or on extendable winches may drop down from locations above a growth environment, and physically manipulate the growth environment or growing aerial mycelium material at one or more specific locations.

### Example 20 Passive Physical Intervention

In yet a further example of the invention, more passive interventions during growth may be employed to obtain a desired material having particular material physical attributes.

For instance, targeted pooling of water droplets may be employed to induce specific physical features in the growing aerial mycelium material.

### FURTHER EXAMPLES OF PHYSICAL MANIPULATION, STRUCTURAL OR APPARATUS ARRANGEMENTS FOR IMPLEMENTING PHYSICAL MANIPULATION STEPS OR OTHER ACTION STEPS TO IMPLEMENT SUPPLEMENTAL GROWTH CONDITIONS

### Linear Platform for Manipulation of Growing Mycelium Fibers

Current aerial mycelium growth in beds relies on controlling or manipulating the entire growth environment of essentially a large 3D box. While this can be done with success, it cannot be done as precisely as one would desire (say within a 10×10×10 cm space). This better precision would be desirable to influence the growth of aerial mycelium which grows on an order of 0.01×0.01×0.01 mm and also over the temporal domain (t=minutes).

In order to address this need, a local environment optimization, built on the same chassis used by mushroom farms for harvesting mushrooms and otherwise manipulating mushroom beds is now proposed. The core operation relies on an existing tuned aerial mycelium growth environment with a target set of generalized conditions which produce aerial mycelium. This operation is then improved by a machine that runs upon two bed or other support structure side rails or other side support structures of each bed in a linear fashion.

As the machine moves up and down a bed or other growth matrix supporting structure (time period can range from 10 seconds to 10 hours to traverse the length of a bed) it uses various “actuator heads” to manipulate the environment and in some cases, the inoculated substrate, growth matrices, and/or aerial mycelium growing directly beneath it. In this fashion, one is able to more precisely control the local environment of growing aerial mycelium, dynamically change it over time, and implement supplemental growth conditions as desired.

In one example embodiment, the bed runner has an array of 100 1×1 cm fans pointed at a 45-degree angle to the growing mycelium. Each fan is controlled by a computer controller which can vary the rpm from 0-9,0000 RPM. The fast runner travels up and down the bed and using feedback from an image system (image sensors) mounted on the fast runner, compares the surface condition of the aerial mycelium on the bed to the target surface condition. Using the computer fans, it dynamically changes the airflow rate directly above the bed. This either adjusts the bulk environment above the bed, or may be used to directly manipulate the growing aerial mycelium fibers, matting them together, weaving them into strands, or otherwise forcing them to touch and agglomerate (or separate in some cases) as may be desired, apart from the base-line growth conditions.

This same platform (which moves spatially during a run, like a “print head”, leaving traces of physical action of the entire shelf, rack, or portion thereof) may be adopted with other print heads, which can deposit chemicals, nutrients, other materials, fats, solids, or living organisms. They may include fibers or rods or pins that are pushed, pulled, or otherwise impacting the surface, rollers to compress the surface (such rollers to be smooth or textured), cutting, scoring, slicing, or other texturing devices, as well as other tools such as electron beams, lasers, lights for localized impact as desired.

Other equipment, to selectively provide nutrients, moisture, and other solutes through pipes and embedded liquid dispensing systems may be implemented to provide supplemental environmental conditions.

The hardware and data processing apparatus used to implement the various illustrative functions described in connection with the aspects disclosed herein may be implemented or performed with a general-purpose single-or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, e.g., one or more modules of computer program instructions, encoded on a computer storage media for execution by, or to control the operation of, data processing apparatus. If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

A software module for use with the methods or systems may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, include compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

### SCOPE OF DISCLOSURE

It will be understood that although the present disclosure is discussed within the context of food and textiles, the embodiments described herein can be implemented in non-food, or other non-textile applications.

### IMPLEMENTATIONS

In some aspects, the present disclosure provides for an aerial mycelium, and for methods of making an aerial mycelium, wherein the aerial mycelium is a growth product of a fungus. In some embodiments, the fungus is a species of the phylum Basidiomycota, Ascomycota or any of the early branching lineages of fungi, formerly referred to as Zygomycota.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of a feature as implemented.

While certain embodiments of the inventions have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatuses, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of, or combined with, any other aspect described. For example, an apparatus may be implemented, or a method may be practiced using any number of the aspects set forth herein. In addition, the scope of the disclosure is intended to cover such an apparatus or method which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for providing supplemental growth conditions to inoculated substrates for the support of growing aerial mycelium materials, alternatively, to growth matrices for the support of growing aerial mycelium materials, or alternatively to growing aerial mycelium materials themselves, situated on either inoculated substrates or growth matrices, the method comprising:
a) providing a growth environment including at least an inoculated substrate for supporting the growth of aerial mycelium material, alternatively a growth matrix for supporting the growth of aerial mycelium material, or alternatively, growing aerial mycelium material, which growth environment supports base-line growth conditions for the growth of aerial mycelium material;
b) conducting at least one of the following steps upon either of said inoculated substrate, growth matrix, or growing aerial mycelium material in situ within the growth environment,
1. performing a physical manipulation on at least one of the inoculated substrate, growth matrix, or growing aerial mycelium, such that the physical manipulation imparts a physical change in at least one of the inoculated substrate, growth matrix, or growing aerial mycelium, which physical change enhances at least one aerial mycelium material physical attribute when compared with aerial mycelium grown under the base-line growth conditions without the physical manipulation, with the physical change being selected from the group consisting of: selectively displacing substrate, growth matrix, or growing aerial mycelium, selectively depressing substrate, growth matrix, or growing aerial mycelium, selectively compressing substrate, growth matrix, or growing aerial mycelium, selectively layering growing aerial mycelium, selectively separating, texturing, or patterning portions of growing aerial mycelium, selectively aligning fibers of growing aerial mycelium via either direct contact or indirect contact techniques, and selectively creating spatial voids in the growing aerial mycelium,
2. adding chemical, biologic, or other substance to at least one of the inoculated substrate, growth matrix, or growing aerial mycelium, such that the addition enhances at least one growing aerial mycelium attribute selected from the group consisting of improved taste as a result of chemical, biologic, or substance incorporation or uptake from the addition into the structure of the growing aerial mycelium, or deposition on the growing aerial mycelium surface, desired color change as a result of chemical, biologic, or substance incorporation or uptake from the addition into the structure of the growing aerial mycelium, or deposition on the growing aerial mycelium surface, desired improvement in nutritional content for human or animal consumption as a result of chemical, biologic, or substance incorporation or uptake from the addition into the structure of the growing aerial mycelium, or deposition on the growing aerial mycelium surface, desired improvement in shelf-life of final aerial-mycelium based product as a result of chemical, biologic, or substance incorporation or uptake from the addition into the structure of the growing aerial mycelium or deposition on the growing aerial mycelium surface;
c) allowing growing aerial mycelium to continue growing in the growth environment in order to either develop an enhanced physical attribute selected from the group consisting of improved or targeted density, homogeneous or heterogeneous surface topology with limited or spatially directed aerial mycelium bulbous occurrence per given unit area, or more aligned or patterned fiber orientation, or to develop an enhanced product performance attribute selected from improved taste, shelf-life, texture, mouth-feel, and nutritional content.

2. The method of claim 1, wherein said grown aerial mycelium that has experienced either in situ physical manipulation or chemical, biologic, or substance addition to either a substrate, growth matrix, or growing aerial mycelium itself, is optionally terminated (in that the aerial mycelium organism is rendered inert) and/or separated from the substrate or growth matrix.

3. The method of claim 1, wherein at least one physical manipulation is performed on the growing aerial mycelium in situ.

4. The method of claim 1, wherein at least two physical manipulations are performed on the growing aerial mycelium in situ.

5. The method of claim 1, wherein at least one chemical, biologic, or substance addition is performed on either the inoculated substrate, growth matrix, or growing aerial mycelium in situ.

6. The method of claim 1, wherein at least two chemical, biologic, or substance additions are performed on either the inoculated substrate, growth matrix, or growing aerial mycelium in situ.

7. The method of claim 1, wherein at least one physical manipulation and one chemical, biologic, or substance addition is performed on at least one of the inoculated substrate, growth matrix, or growing aerial mycelium in situ.

8. The method of claim 1, wherein at least one physical manipulation and one chemical, biologic, or substance addition is performed on at least one of the inoculated substrate, growth matrix, or growing aerial mycelium in situ concurrently in time, such that there is at least some overlap in time that such physical manipulation and chemical, biologic, or substance addition occurs.

9. The method of claim 1, wherein there is no overlap in time that at least one physical manipulation and one chemical, biologic, or substance addition is performed on at least one of the inoculated substrate, growth matrix, or growing aerial mycelium in situ.

10. The method of claim 1, wherein at least one physical manipulation and one chemical, biologic, or substance addition is performed sequentially on at least one of the inoculated substrate, growth matrix, or growing aerial mycelium in situ.

11. The method of claim 1, wherein at least two physical manipulations are performed on at least one of the inoculated substrate, growth matrix, or growing aerial mycelium in situ, wherein the at least two physical manipulations are separated temporally (by a period of time) in which temporal gap there are either no physical manipulations occurring, or there are different physical manipulations occurring from those of the at least two physical manipulations.

12. The method of claim 1, wherein two of the same physical manipulations are performed on at least one of the inoculated substrate, growth matrix, or growing aerial mycelium in situ, but such physical manipulations are separated temporally by a time period of no physical manipulation.

13. The method of claim 1, wherein at least two chemical, biologic, or substance additions are performed on at least one of the inoculated substrate, growth matrix, or growing aerial mycelium in situ, wherein the at least two chemical, biologic, or substance additions are separated temporally (by a period of time) in which there are either no chemical, biologic, or substance additions occurring in the temporal gap, or there are different chemical, biologic, or substance additions occurring from those of the at least two chemical, biologic, or substance additions.

14. The method of claim 1, wherein two of the same chemical, biologic, or substance additions are performed on at least one of the inoculated substrate, growth matrix, or growing aerial mycelium in situ, but such chemical, biologic, or substance additions are separated temporally by a time period in which there is no chemical, biologic, or substance addition.

15. The method of claim 1, wherein a temporal period occurs between a multiplicity of either physical manipulation or chemical, biologic, or substance additions, in which either physical manipulation or chemical, biologic, or substance addition occurrences are separated by at least one temporal period in which either physical manipulation or chemical, biologic, or substance addition does not occur, and the growing aerial mycelium is left undisturbed to grow without supplemental physical manipulation or chemical, biologic, or substance addition.

16. The method of claim 1, wherein the chemical, biologic, or substance addition in situ includes a material which imparts animal meat-like taste to the aerial mycelium.

17. The method of claim 16, wherein the chemical, biologic, or substance addition in situ which imparts animal meat-like taste to the aerial mycelium reacts in the presence of a sugar and heat to form such taste.

18. The method of claim 16, wherein the chemical, biologic, or substance addition in situ which imparts animal meat-like taste to the aerial mycelium is thiamine.

19. The method of claim 1, wherein the physical manipulation in situ creates either a homogeneous or heterogeneous surface topology of the growing aerial mycelium.

20. The method of claim 1, wherein the physical manipulation in situ is exposure to sound waves.

21. The method of claim 1, wherein the physical manipulation is a direct contact with an apparatus in situ that either depresses, compresses, slices, cuts, scores, layers, abrades, scratches, creates voids, repositions portions of the aerial mycelium, or a combination thereof.

22. The method of claim 1, wherein at least one physical manipulation of the aerial mycelium in situ is accompanied by at least one chemical, biologic, or other substance addition in situ.

23. The method of claim 1, wherein at least one physical manipulation is performed on the growing aerial mycelium in situ.

24. The method of claim 1, wherein at least one chemical, biologic, or other substance addition is added to the growing aerial mycelium in situ.

25. The method of claim 1, wherein at least one physical manipulation and/or at least one chemical, biologic, or other substance addition is performed in situ on the growing aerial mycelium, resulting in at least one enhanced aerial mycelium attribute selected from the group consisting of increased density, increased fiber alignment, targeted taste, targeted texture, increased surface homogeneity, increased surface heterogeneity, enhanced color, enhanced mouth feel, enhanced tear strength, enhanced tensile strength, enhanced elasticity and enhanced void space in the aerial mycelium (especially when compared with that achieved under base-line growth conditions without such supplemental condition change of either physical manipulation and/or at least one chemical, biologic, or other substance addition).
